(19) United States
(12) Patent Application Publication
(10) Pub. No.: US 2022/0037691 A1
(43) Pub. Date: Feb. 3, 2022

(54) INTERCONNECTED PHOTOSYNTHESIS MATRIX AND BIO-ENERGY PRODUCTION SYSTEMS

(71) Applicant: Kohane Technologies, LLC, Chanhassen, MN (US)

(72) Inventors: Michael Kohane, Chanhassen, MN (US); Matthew Kohane, Chanhassen, MN (US)

(73) Assignee: Kohane Technologies, LLC, Chanhassen, MN (US)

(21) Appl. No.: 17/503,633

(22) Filed: Oct. 18, 2021

Related U.S. Application Data
(63) Continuation-in-part of application No. PCT/US2020/028873, filed on Apr. 17, 2020.
(60) Provisional application No. 62/835,891, filed on Apr. 18, 2019.
(60) Provisional application No. 63/149,097, filed on Feb. 12, 2021.
(60) Provisional application No. 63/251,231, filed on Oct. 1, 2021.

(51) Int. Cl. H01M 8/16; C12M 1/34; C12M 1/00

(52) U.S. Cl. CPC H01M 8/16; C12M 21/00; C12M 41/00

(57) ABSTRACT

An interconnected photosynthesis matrix and bio-energy production system. More specifically, a self-sustaining bio-system that uses the bio-energy production system, which comprises a selection process, an extraction process, and a transfer process, to create an energy enhanced organism and then uses the energy from the energy enhanced organisms for human use and/or for the second portion of the system, the photosynthesis matrix, where photosynthesis takes place. The energy is extracted from the energy enhanced organism by creating an energy rich homogenate, and then the energy is transferred to the grid, to an energy storage device, or to the photosynthesis matrix. The photosynthesis matrix consumes carbon dioxide and reduces carbon dioxide concentration while producing glucose, which it then provides to the bio-energy production system. The two systems work together in a feedback loop to allow continuous chemical reactions.

### CROSS REFERENCE TO THE RELATED APPLICATIONS

This application is a continuation-in-part of International Patent Application No. PCT/US2020/028873, filed Apr. 17, 2020, which claims benefit of U.S. Provisional Application No. 62/835,891, filed Apr. 18, 2019, and additionally claims priority to U.S. Provisional Patent Application No. 63/149,097, filed Feb. 12, 2021, and U.S. Provisional Patent Application No. 63/251,231, filed Oct. 1, 2021, the entire disclosures of which are hereby incorporated by reference.

### FIELD OF THE DISCLOSURE

The disclosed invention generally relates to bio-energetics. More specifically, the disclosed process and method involves increasing carbon dioxide reduction rates, enhancing primary bio-energy systems, such as fruit fly colonies, and harvesting a high yield of electrons, protons, and ATP from the enhanced bio-energy system. The harvested energy can then immediately be made available, or it can be stored for future human use.

### BACKGROUND OF THE INVENTION

Carbon dioxide is a natural byproduct of life on Earth and has several sources such as animal respiration, decaying organic products (plants, animals, etc.), automobiles and industry, and others. Currently, human energy derivation methods are causing increased incremental imbalances within the atmosphere and ecosystems of our planet. More specifically, as the human population continues to expand, carbon dioxide emissions continue to increase at a rate faster than that at which the carbon sinks can absorb it. Since the amount of carbon dioxide in the atmosphere directly impacts the Earth's temperature, an increase in carbon dioxide sources without a direct increase in sinks will lead to negative environmental effects. Since the increase of the human population does not appear to be slowing down, a method for increasing the rate and/or sources of carbon sinks is needed.

Examples of alternative energy technologies in relatively widespread use are onshore wind, offshore wind, conventional turbine, combined cycle turbine, geothermal, solar PV, hydroelectric, solar thermal, CSP, biomass, biofuel, nuclear, and coal. However, these forms of energy also have major drawbacks. They place physical demand upon the environment in the form of the materials that are physically needed. These methods are also intermittent, the sun may not shine bright nor will the wind gust constantly. Additionally, the energy harvested from renewable sources often cannot be stored and/or can only be transferred at great loss of the harvested energy. Therefore, it must often immediately be transferred to the grid. A source of safe, renewable, and environmentally friendly energy is needed that is energy rich, can be produced at one locality, and can be stored and made available as it is needed.

### SUMMARY OF THE INVENTION

Generally, three disclosed systems are discussed herein. The first is a system that increases energy availability through a genetic selection process and preparation of an energy rich homogenate from larvae. The second is a system that reduces carbon dioxide through the use of plant extract and unicellular photosynthesis flagellates. In some cases, ATP may be added to the second system to expedite the rate that carbon dioxide concentrations are reduced by the system. The third is a combined system that improves photosynthesis in the second system using ATP from the energy rich homogenate from the first system. The first system provides ATP, an energy requirement for photosynthesis. The second system provides glucose, an energy requirement for the larvae used to prepare the energy rich homogenate. In some embodiments, system three can be a self-regenerating, perpetuating, bioenergy system that consumes excess carbon dioxide in the atmosphere and produces energy for human use.

The disclosed interconnected photosynthesis matrix and bio-energy production systems can consume carbon dioxide at an increased rate and can increase bio-energy availability for human use through a technique that does not harm humans or the environment. Additionally, the bio-energy can be generated in one location. The system is based on a renewable biological process (selected, breeding fruit fly strains) that primarily store high levels of energy. This energy can be made available for human use as needed in an efficient way that is analogous to fossil fuels and independent of environmental conditions.

The bio-energy production system described herein involves three main components: (1) creation of energy-enhanced organisms using selection pressures; (2) extraction of energy from the energy-enhanced organisms by creating an energy rich homogenate, which can be supplemented with NAD; (3) transfer of the extracted energy to a device such as an energy grid or a storage device.

A complimentary photosynthesis matrix is also proposed that reduces atmospheric carbon dioxide levels and produces glucose, thereby subsequently driving the fruit fly bio-energy production system. More specifically, the matrix can be comprised of a plant/chloroplast extract and can consume atmospheric carbon dioxide and produce glucose, oxygen, and water. The glucose that is produced can, in turn, be fed to the fruit fly strains and also integrated with, or supplemented to, the energy-rich homogenate. The selected fruit fly strains and the energy-rich homogenate that is supplemented with NAD can then produce ATP, and that ATP can be added to the photosynthesis matrix to help drive the photosynthesis in that matrix. In some cases, the bio-energy production system can be directly added to the photosynthesis matrix producing enhanced reaction rates and a self-sustaining system.

In one aspect, the disclosure provides an interconnected photosynthesis matrix and bio-energy production system, the interconnected system comprising: a bio-energy production system having a selection process and an extraction process; and a photosynthesis matrix having carbon dioxide and a chloroplast solution. In some embodiments, the selection process can be applied to a first organism strain for a plurality of generations to create a second organism strain with enhanced energy availability, and the extraction process can create an energy rich homogenate from the energy enhanced organism strain. The chloroplast solution can include homogenized plant material and an ATP solution, and the ATP solution can be derived from the energy rich homogenate.

In some embodiments, the bio-energy production system can create electrons and protons for human use. Additionally, the photosynthesis matrix can consume the carbon dioxide and produce glucose. Further, the photosynthesis matrix can be housed on at least one drone, the drone including wires that attract additional carbon dioxide molecules.

In some embodiments, the glucose from the photosynthesis matrix can be used as a food source for the bio-energy production system. Additionally, the ATP solution can be used by the photosynthesis matrix at the same rate that the glucose is used by the bio-energy production system. Further, in some cases, the glucose can be used by the bio-energy production system during the selection process.

In some embodiments, the interconnected system may be incorporated into a passive cellular array. Some versions of the passive cellular array may include a set of cells, wherein each cell in the passive cellular array includes both the energy rich homogenate solution and the chloroplast solution. In other versions, each cell in the passive cellular array may include either the energy rich homogenate solution or the chloroplast solution. Further, each energy rich homogenate solution cell can be surrounded by chloroplast solution cells and each chloroplast solution cell can be surrounded by energy rich homogenate solution cells. Therefore, each cell can interface with each adjacent cell to transfer output elements, and the output elements may include ATP from the energy rich homogenate solution cells and glucose from the chloroplast solution cells.

In some embodiments, the interconnected system may be incorporated into an active array. The active array can include a plurality of active chambers, a capture chamber, a holding chamber, and a redistribution chamber. Each active chamber can contain the energy rich homogenate, the chloroplast solution, or a combined solution having both. The capture chamber can capture ATP, electrons, and protons from each chamber having the energy rich homogenate and glucose from each chamber having the chloroplast solution. The holding chamber can accept and hold ATP, electrons, protons, and glucose for future use. The redistribution chamber can accept and redistribute ATP, electrons, protons, and glucose to other chambers within the active array. Each cell in the active array may further include a Clark chamber that reduces the connection distance between each of the other chambers.

In some embodiments, the interconnected system can be incorporated into an assembly of chambers and sensors that are connected by pumps. An energy rich homogenate chamber may be connected to a filter chamber, the filter chamber can connect to a first biomolecule sensor, the first biomolecule sensor can connect to a photosynthesis chamber, the photosynthesis chamber can connect to a second biomolecule sensor, and the second biomolecule sensor can connect to the energy rich homogenate chamber. The photosynthesis chamber may have at least one perforated, elongate chamber covered by a clear tube and having chloroplast dispersed within. Additionally, an input reservoir can connect to the energy rich homogenate chamber, a coulochem cell can connect in between the energy rich homogenate chamber and the filter chamber, the first biomolecule sensor can connect to the energy rich homogenate chamber, a carbon dioxide input can connect to the photosynthesis chamber and add carbon dioxide to a first end of the photosynthesis chamber, and peristaltic pumps can move material between chambers, sensors, the coulochem cell and the input reservoir.

In another aspect, the disclosure provides a method for reducing carbon dioxide, the method comprising creating an energy enhanced organism strain by using a selection process applied to a first organism strain for a plurality of generations; creating an energy rich homogenate from the energy enhanced organism strain; pumping the energy rich homogenate from an energy rich homogenate chamber into a filter chamber to produce a filtered material; pumping the filtered material through a first biomolecule sensor to a photosynthesis chamber; adding a predetermined quantity of carbon dioxide to an inferior end of the photosynthesis chamber; combining the filter material with the carbon dioxide and a chloroplast solution in the photosynthesis chamber; reducing the quantity of carbon dioxide within the photosynthesis chamber through photosynthesis; creating a glucose product within the photosynthesis chamber; and pumping at least the glucose product from a superior end of the photosynthesis chamber through a second biomolecule sensor and to the energy rich homogenate chamber.

In another aspect, the disclosure provides an interconnected photosynthesis matrix and bio-energy production system, the interconnected system comprising: a bio-energy production system having a selection process and an extraction process; and a photosynthesis matrix having carbon dioxide and a chloroplast solution. In some embodiments, the selection process can be applied to a first organism strain for a plurality of generations to create a second organism strain with enhanced energy availability, and the extraction process can create an energy rich homogenate from the energy enhanced organism strain. The chloroplast solution can include homogenized plant material and an ATP solution, and the ATP solution can be derived from the energy rich homogenate. The photosynthesis matrix can consume the carbon dioxide and produce glucose and the glucose from the photosynthesis matrix can be used as a food source for the bio-energy production system. The interconnected system can be incorporated into an assembly of chambers and sensors that are connected by pumps, and one of the chambers can be a photosynthesis chamber. The photosynthesis chamber can include of a plurality of perforated, elongate chambers that are each covered by a clear tube and have chloroplast dispersed within. The plurality of perforated, elongate chambers can connect to a carbon dioxide input on a first end.

### BRIEF DESCRIPTION OF THE DRAWINGS

FIG. 1 is a diagram of carbon flow and electron flow in metabolism.

FIG. 2 illustrates an embodiment of a system that makes bio-energy available for human use.

FIG. 3 illustrates one embodiment of the extraction system.

FIG. 4 is a diagram depicting feedback loops in one embodiment of the current disclosure.

FIG. 5 is a diagram depicting how energy extracts from an organism can be coupled to various technologies for human use after selecting for enhanced energy availability in the organism.

FIG. 6 is a diagram depicting an overall selection process for enhanced energy availability in an organism and further depicting the various ways the selected organisms can be processed for human use.

FIG. 7 is a diagram depicting an instrument configuration for the disclosed system.

FIG. 8 is a diagram depicting one embodiment of a selection process for the disclosed system.

FIG. 9 is a diagram depicting a process for creating a homogenate from an organism.

FIG. 10 is a diagram depicting carbon flow and electron flow that occurs in the disclosed system after extraction of energy from an organism.

FIG. 11 is a diagram depicting the various ways an energy rich homogenate can be manipulated for human energy use or storage.

FIG. 12 is a diagram depicting extraction of energy from an organism.

FIG. 13 is a diagram depicting the various energy rich homogenates that are created by using the disclosed system and how those homogenates can be used as energy for human use.

FIG. 14 is a diagram depicting one embodiment of a selection process for the disclosed system.

FIG. 15 is a diagram depicting one embodiment of a selection process for the disclosed system.

FIG. 16 is a schematic depicting the atmospheric impact of the interconnected photosynthesis matrix and bio-energy production systems.

FIG. 17 is a schematic depicting the positive feedback loop between the photosynthesis matrix and the bio-energy production system.

FIG. 18 is a schematic depicting the chemical processes taking place in each of the two main systems.

FIG. 19 is an illustrative diagram showing each of the components for the experimental photosynthesis matrix.

FIG. 20 is an illustrative diagram showing each of the components for the experimental photosynthesis matrix in addition to a glucose output for the bio-energy production system.

FIG. 21 is a schematic depicting the chemical processes taking place at each step in the interconnected systems.

FIG. 22 is a schematic of the photosynthesis substrate, mechanism, and matrix physically combined with the fruit fly substrate, mechanism, and matrix.

FIG. 23 illustrates a scalable model of the photosynthesis matrix.

FIG. 24 illustrates how the disclosed photosynthesis matrix can utilize drone technology.

FIG. 25 illustrates the movement of electrons along the electron transport chain as applied to the energy rich homogenate solution.

FIG. 26 illustrates an example cellular array incorporating sunlight.

FIG. 27 illustrates an example cellular array incorporating sunlight, wherein the cells are arranged to interface with each other.

FIG. 28 illustrates an example cellular array incorporating sunlight, wherein the cells are arranged to interface with each other.

FIG. 29 illustrates an example cellular array incorporating sunlight, wherein the cells are arranged to interface with each other.

FIG. 30 illustrates an example cellular array incorporating sunlight, wherein the cells are arranged to interface with each other.

FIG. 31 illustrates an example cellular array incorporating sunlight, wherein the cells are arranged to interface with each other.

FIG. 32 illustrates a cellular interface wherein two cells are shown interfacing with each other.

FIG. 33 illustrates an example of advanced design incorporating specific chambers and solutions with interface and exchange.

FIG. 34 illustrates an example of advanced design incorporating specific chambers and solutions with interface and exchange.

FIG. 35 illustrates an example of advanced design incorporating specific chambers and solutions with interface and exchange.

FIG. 36 illustrates a first embodiment of a master assembly diagram for an interconnected photosynthesis matrix and bio-energy production system.

FIG. 37 illustrates a rear view of the first embodiment of the master assembly diagram for an interconnected photosynthesis matrix and bio-energy production system.

FIG. 38 illustrates a second embodiment of a master assembly diagram for an interconnected photosynthesis matrix and bio-energy production system.

FIG. 39 illustrates a third embodiment of a master assembly diagram for an interconnected photosynthesis matrix and bio-energy production system.

FIG. 40 illustrates an example of an assembly of the energy rich homogenate chamber having inner coils.

FIG. 41 illustrates an example of an assembly of the photosynthesis chamber.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims. It is understood that various omissions and substitutions of equivalents are contemplated as circumstances may suggest or render expedient, but these are intended to cover applications or embodiments without departing from the spirit or scope of the claims attached hereto. Also, it is to be understood that the phraseology and terminology used herein are for the purpose of description and should not be regarded as limiting.

### General Overview

The disclosed interconnected photosynthesis matrix and bio-energy production systems are self-sustaining bio-systems that, using a first portion of the system herein referred to as the “bio-energy production system,” can produce energy in biological organisms, such as fruit flies. This energy can be intended for human use and/or can be allocated to a second portion of the system herein referred to as the “photosynthesis matrix,” where photosynthesis takes place. Using the photosynthesis matrix, the system can reduce carbon dioxide levels and provide glucose for the bio-energy production system (for example, glucose can be provided during the selection process when creating an energy enhanced fruit fly strain). The two systems can work together in a feedback loop to allow continuous chemical reactions.

Overview of the Bio-Energy Production System

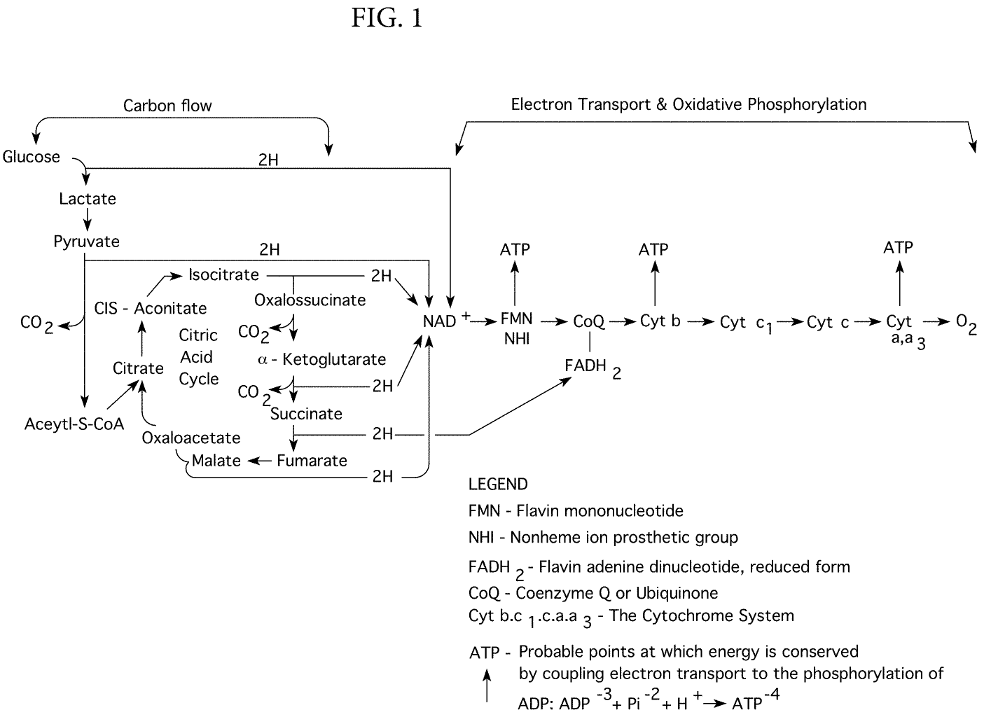

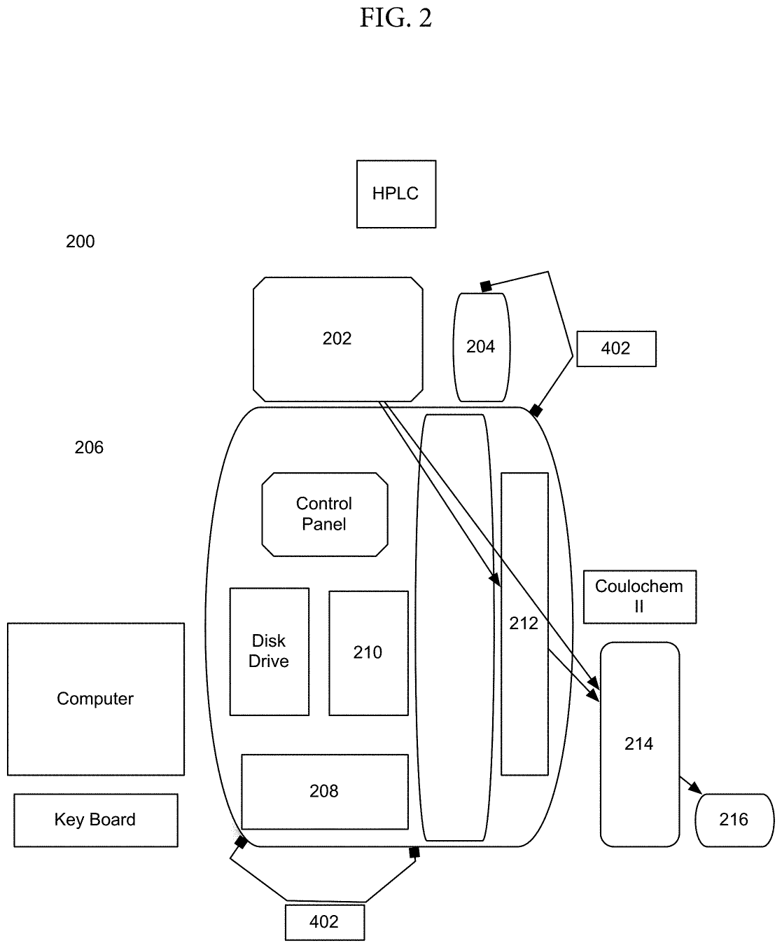

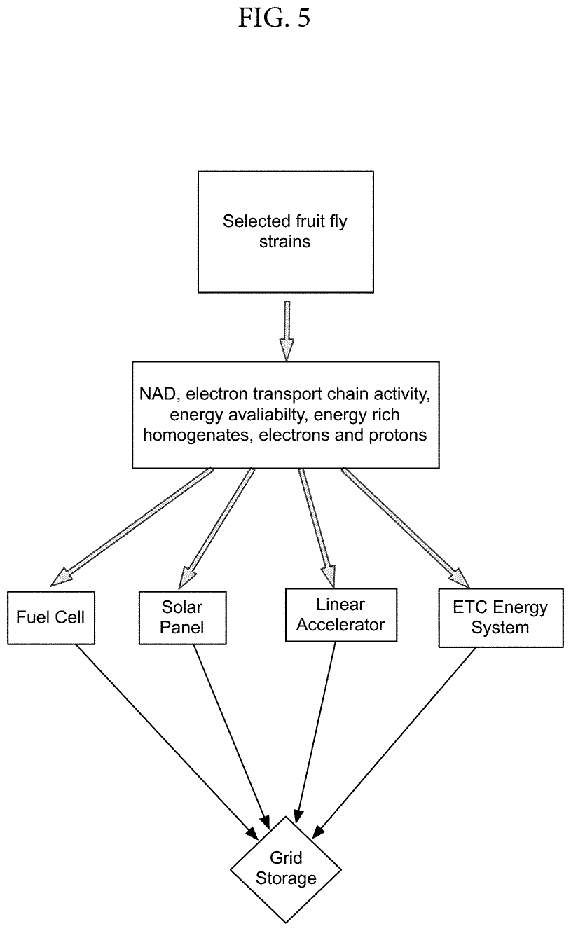

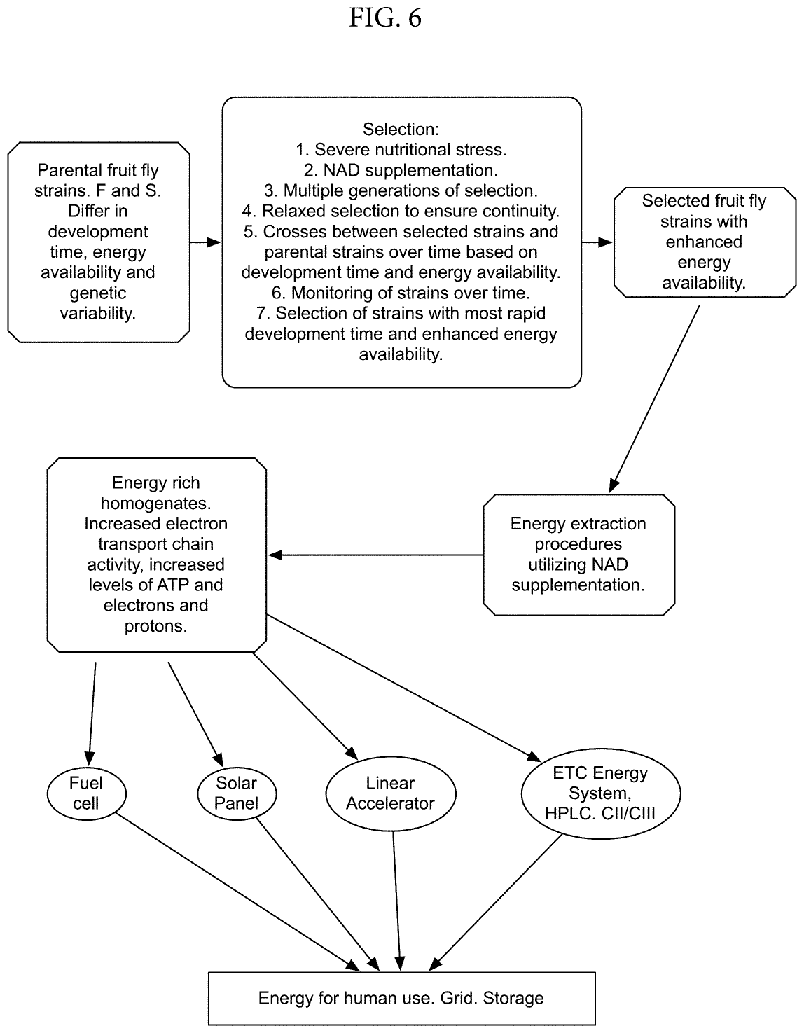

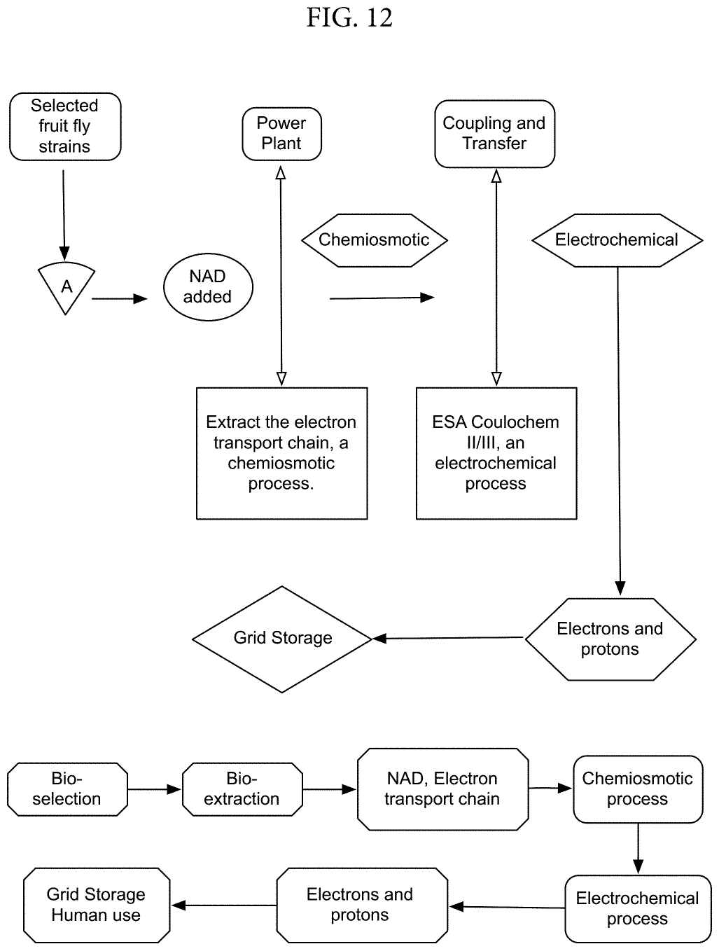

The disclosed invention describes a method and process for harvesting, transferring, and storing energy from biological, carbon-based material to provide energy for human use. FIG. 1 illustrates the central role of NAD in energy metabolism. Specifically, it illustrates the central role in the collection of electrons in energy metabolism and the transfer of electrons to the electron transport chain (ETC). In general, the disclosed system, illustrated in FIGS. 5-6 and 12, transfers electrons and protons from a biological organism to the grid or to a storage device. After selection of an energy enhanced fruit fly strain, an energy rich homogenate can be created and coupled directly to fuel cells, solar panels (PVS), a linear accelerator, or to an electron transport chain (ETC) energy system, as illustrated in FIGS. 2-3, 7, and 12.

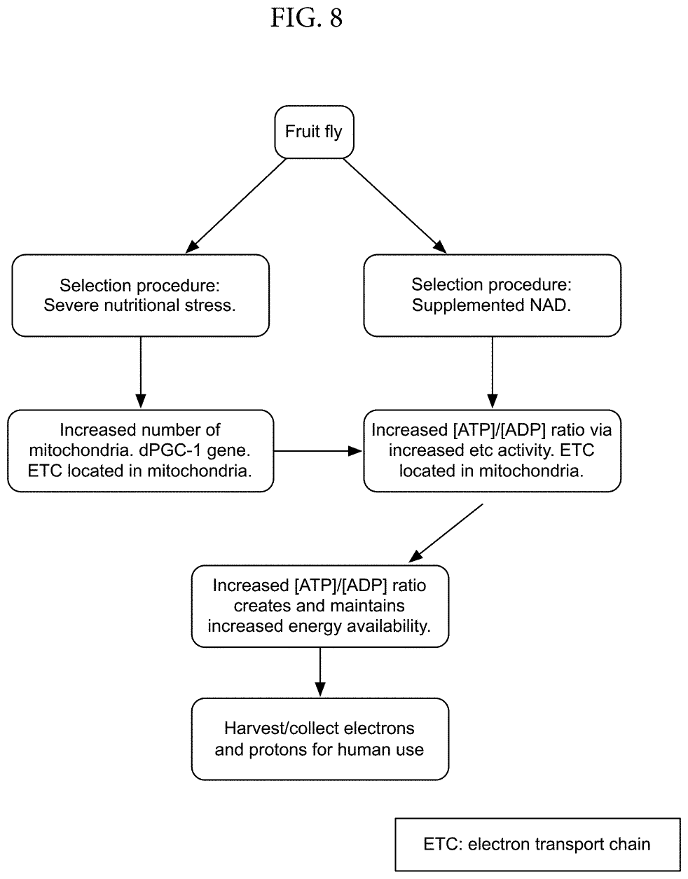

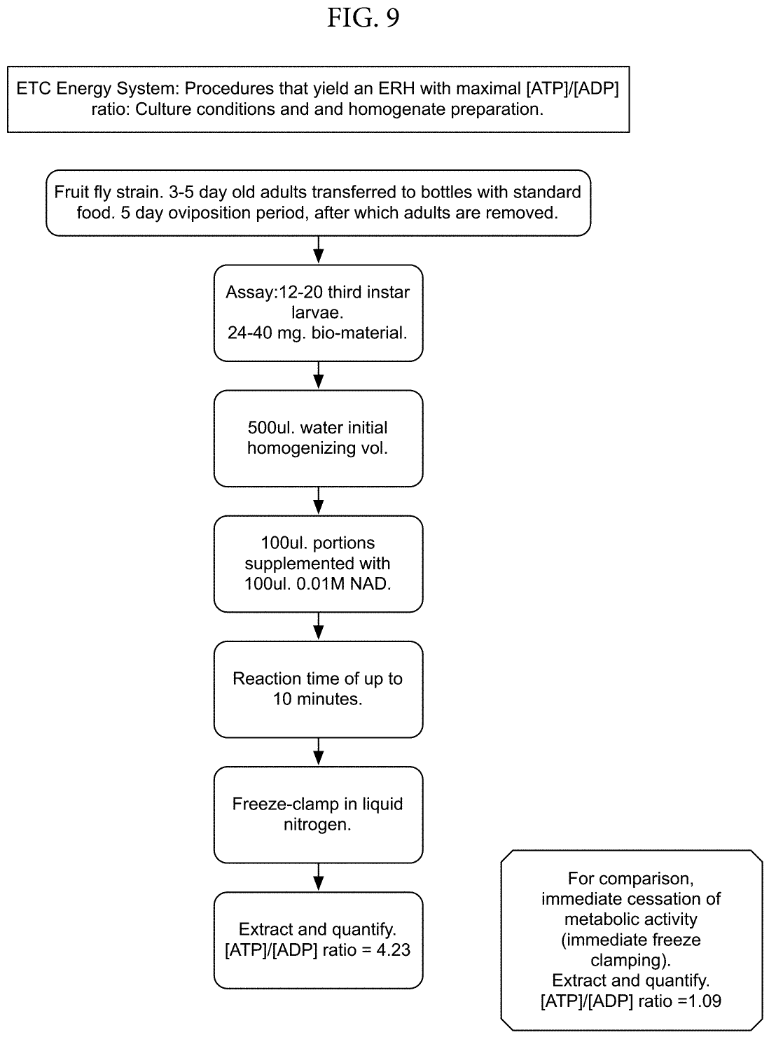

Therefore, the bio-energy production system includes a selection process, briefly illustrated in FIG. 8, extraction process, illustrated in FIG. 9, and transfer process providing immediate energy available for human use or direct access to the grid and storage devices. The selection process, generally, involves placing selection pressures on biological organisms to enhance their energy availability. These selected biological organisms will be the primary storage of energy. The extraction process, generally, involves extracting energy, in the form of electrons, protons and ATP, from the biological organisms. The transfer process, generally, involves transferring the energy from the biological organisms and either (1) providing immediate energy available for human use or (2) transferring the energy to the grid or a storage device.

Selection Process

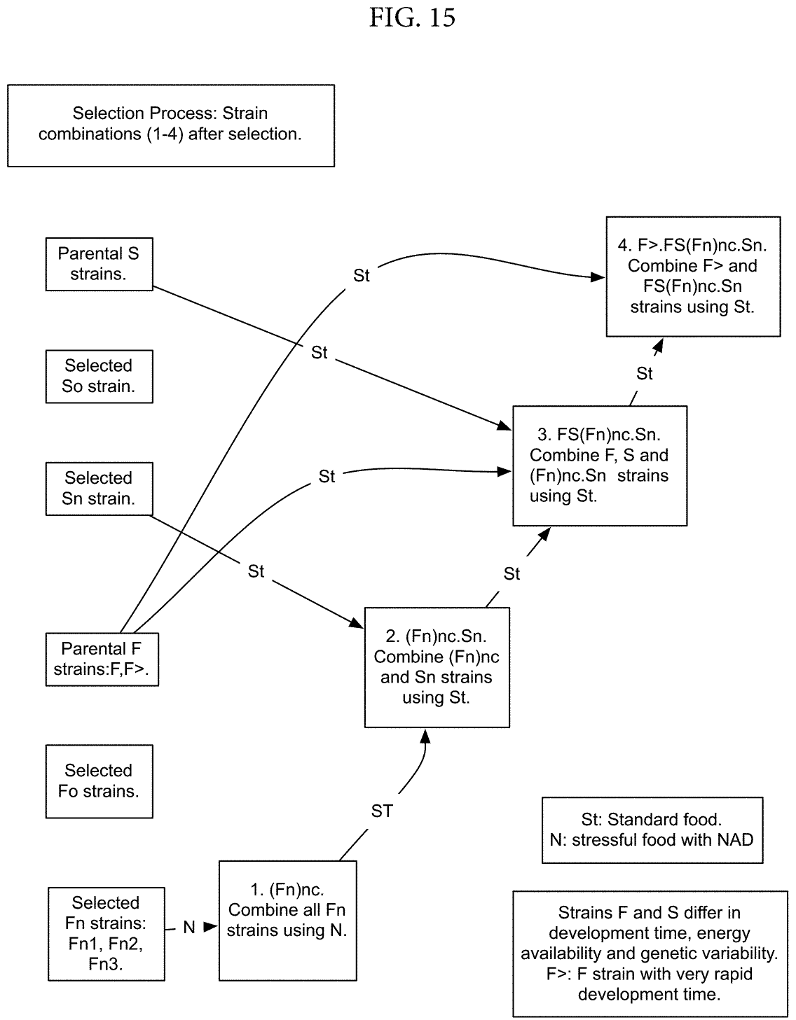

In some embodiments, the disclosed selection process, illustrated in FIGS. 8, 14 and 15, involves using two strains of an organism as the bio-energy source, each strain having different development times (for example, using two Drosophila melanogaster strains: strain F (fast development time) and strain S (slow development time)); severe nutritional stress; continuous multiple generations of selection; use of supplemental NAD and the target of selection (ex: the Electron Transport Chain); relaxed selection to ensure generational continuity; use of the “vac” system, which is a culture in a Faraday cage with specific EMFs; crosses between selected strains and parental strains in various permutations based on decreased development time and increased energy availability; monitoring of selected strains, parental strains, and combined strains over time; and determination of strains with decreased development time and increased energy availability. The selected fruit fly strains can then act as the primary source of energy storage.

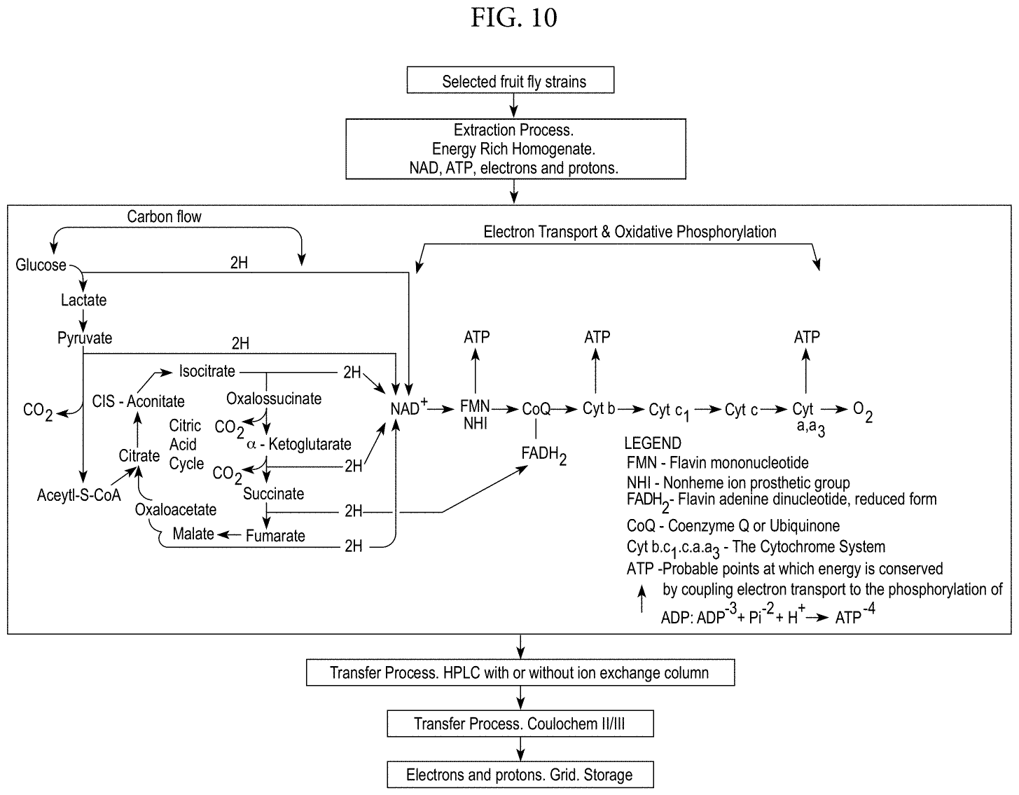

Because stress exposes natural genetic variation, it can be used as a tool to look for variation in energy metabolism and energy availability via the selective agent NAD. The purpose of the disclosed selection process is to create organisms that have an increase in bio-energy availability by exposing the organisms to stressful food conditions. FIG. 1 illustrates the energy flow in metabolism. FIG. 10 illustrates the metabolic energy flow in the disclosed system. Nutrients, such as carbon or glucose, are consumed by the system and, when metabolized, the co-enzyme nicotinamide adenine dinucleotide (NAD) is available. NAD is a direct participant in the ETC where ATP (i.e., energy) is produced, as illustrated in FIG. 1. Therefore, during larval development of fruit flies, supplemental NAD can increase the proportion of ATP available and can increase the ATP/ADP ratio, as illustrated in FIG. 8.

The disclosed selection process is a novel, stress-selection-stabilization model that produces biological material with enhanced bio-energy availability. The selection process can utilize the entire genome of the biological organism and population level processes with no mutagenesis or cloning.

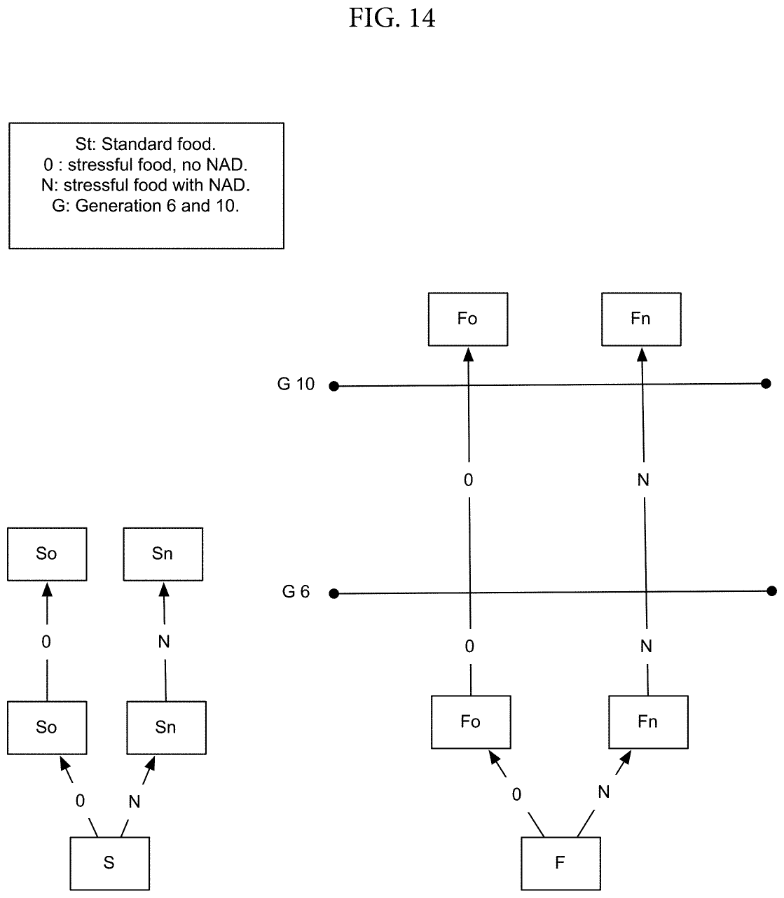

In one embodiment of the selection process, two fruit fly strains, F and S, are used for selection, as illustrated in FIG. 14, to increase genetic diversity and, therefore, enhance energy availability. The two strains can differ in development time, energy availability, and genetic variability. Intense selection for increased energy availability can be carried out for a number of generations (for example, G1 through G5), utilizing, where necessary, relaxed selection to maintain population continuity. Because an increase in energy availability, the ATP/ADP ratio, and ETC activity leads to a decrease in development time, changes in development time can be used as an indicator of increased energy availability.

More specifically, the parental strain of adult flies can be cultured on stressful food supplemented with NAD and removed after their eggs have been laid. Stressful food can include water, yeast, and agar. Once the offspring hatch from the eggs in the stressful food supplemented with NAD, those emerging flies (“G1”) can then be collected and cultured on standard food and removed from the standard food culture after their eggs have been laid. Standard food can be instant dry food and water. The G1 flies have now been hatched on stressful food supplemented with NAD, have been relocated to a standard food culture, and have laid eggs on standard food. When they are removed from the standard food culture, they are placed back on the stressful food supplemented with NAD to lay eggs in that culture. If none of those G1 adults survive, the emerging flies from the standard food condition, the offspring of G1, can then be used as substitutes for G1 to establish the next generation of selection by being placed on the stressful food supplemented with NAD. However, if any of the G1 adults survive, they will be kept on the stressful food supplemented with NAD until they lay eggs, at which point in time they will then be removed. The emerging flies (“G2”) will then complete the same process of the G1 flies, wherein once they hatch, they will be removed to the standard food culture to lay eggs and then transferred back to the stressful food supplemented with NAD to lay eggs, which, if they hatch, become the G3 flies.

In one embodiment, each generation of adults can be given a two-day oviposition period on the stressful food supplemented with NAD. These adults can then be removed and the vials of experimental food can be cultured at 18 degrees Celsius. When all surviving offspring from the experimental food vials have been collected, they can be transferred to a standard food vial for 24 hours. These offspring can then be transferred to the experimental food for a two-day oviposition period to establish the next generation. If there are too few surviving offspring from the experimental food, other progeny of the surviving adults, held on the standard food for 24 hours, can be used to re-establish the offspring generation held on experimental food.

Therefore, the parent generation (G0) can have offspring (G1) that hatch on stressful food supplemented with NAD; G1, once hatched, are then moved to standard food; G1 lays “back-up” eggs on standard food; G1 is moved back to stressful food supplemented with NAD; G1 lays eggs on stressful food supplemented with NAD; G1 is removed from stressful food supplemented with NAD; G1 offspring hatch on the stressful food supplemented with NAD (these offspring being referred to as G2) and are moved to standard food; G2 lays “back-up” eggs on standard food; G2 is moved back to stressful food supplemented with NAD; G2 lays eggs on stressful food supplemented with NAD; G2 is removed from stressful food supplemented with NAD; G2 offspring hatch on the stressful food supplemented with NAD (these offspring being referred to as G3) and are moved to standard food; G3 lays “back-up” eggs on standard food; G3 is moved back to stressful food supplemented with NAD; G3 lays eggs on stressful food supplemented with NAD; G3 is removed from stressful food supplemented with NAD, etc.

Each generation can be conditionally selected based on the ability of the initial surviving flies to establish the next generation. This process ensures continuity of the energy selection process and preserves the changes in energy metabolism and changes in the underlying genetic structure. In one embodiment, after five or six generations, successful strains will appear in the selection process and can be used for the remaining selection process (for example, G5 through G10, G6 through G10, or G6 through G11).

After completion of the selection process, the parental and selection strains with the greatest bio-energy availability can be maintained on standard food, and strain performance can be monitored. Decreased larval development time in the presence of NAD (for example, decreasing from 12.5 days to 11 days) can be attributed to increased bio-energy availability.

Successful strains that exhibit enhanced bio-energy availability can be selected and combined with other successful strains. For example, several successful selection strains can be combined to create a new strain. Alternatively, only two successful selection strains may be combined to create a new strain.

The new strain can then be combined with a selected strain from a different line (example, three strains that started and evolved from strain F can be combined with a strain that started and evolved from strain S). This new strain can then be combined with both parent strains to create a final strain, which is maintained in discrete generations over time. Even though random genetic segregation may occur over time, the final strain can consistently exhibit enhanced energy availability as indicated by decreased development time and large numbers of adult survivors in comparison to all other strains.

In summary, the main steps in the disclosed selection process are to use alternating stressful and non-stressful food conditions on two or more strains of a biological organism, measure energy availability, select strains with enhanced energy availability, stabilize and combine these strains over time, allow the selected strains to vary in energy availability over time as a consequence of population level genetic segregation, combine strains with enhanced energy availability at different times, and select and combine strains that have exhibited enhanced energy availability throughout the timeframe.

Once the desired organism strains have been established, the energy they store can be made accessible to humans in various ways (for example, by using the fruit flies to make an energy rich homogenate). An energy rich homogenate can be made from the fruit flies by using an extraction process, described below and illustrated in FIG. 9. Thereafter, the energy rich homogenate can be used in a fuel cell, a solar panel, a linear accelerator, or an ETC energy system to make the energy available for human use or for transfer to a grid or storage.

Extraction Process

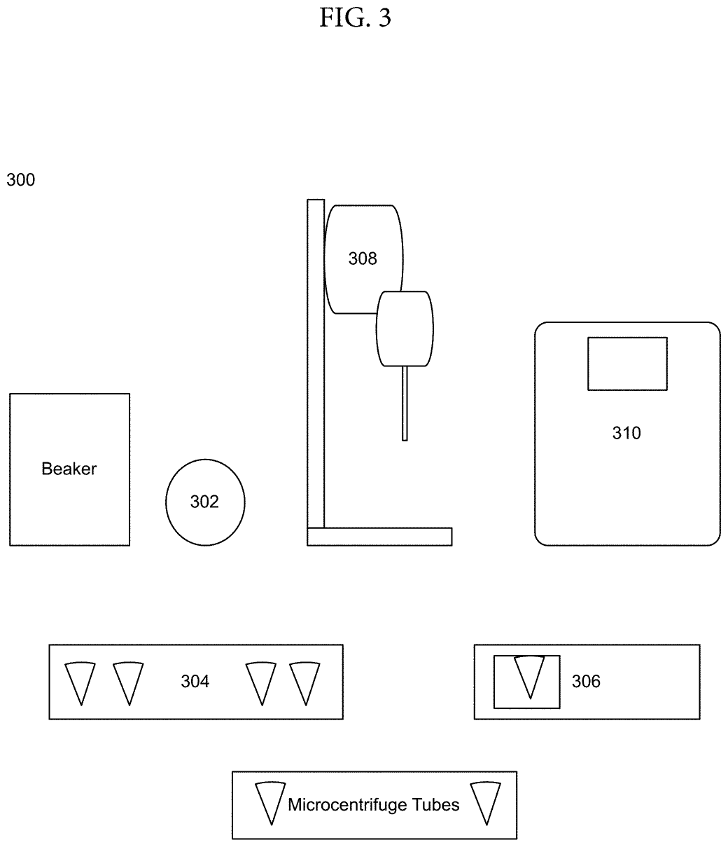

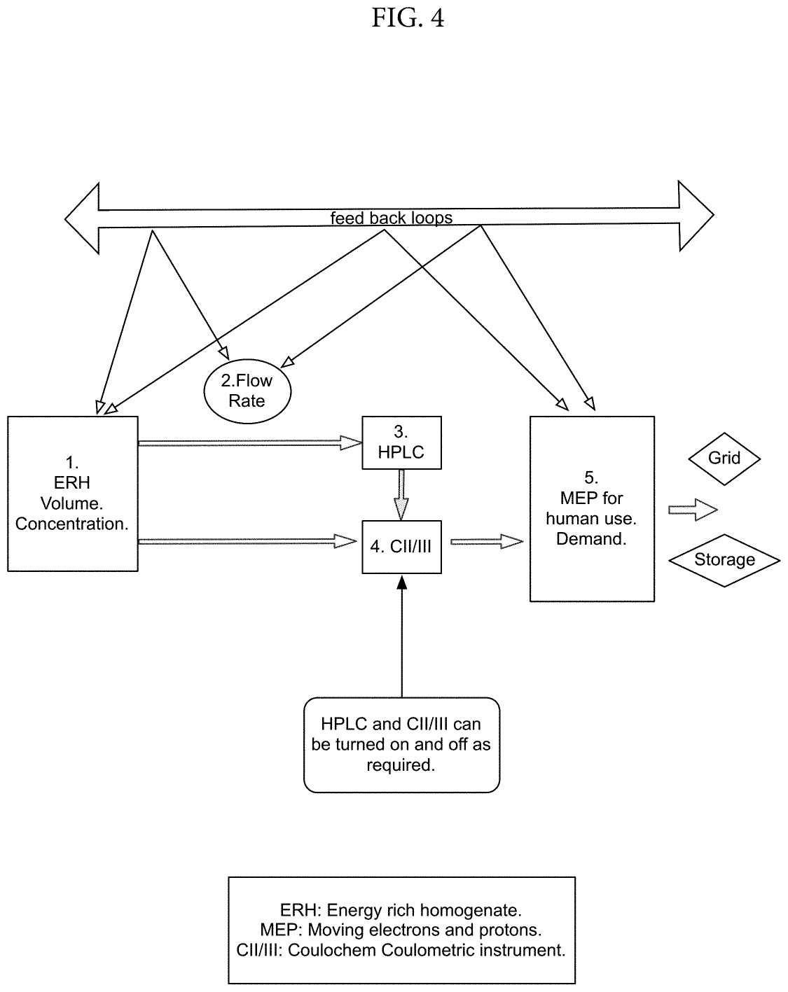

The extraction process, illustrated in FIGS. 9-13 provides energy rich solutions that can be used in a number of ways to provide energy for human use (1) as a component of the fuel cell (2), as a component of the solar panel, (3) in combination with components of the linear accelerator, or (4) by using the ETC energy system, described below. Materials that comprise the extraction system 300 are illustrated in FIG. 3 and can include, but are not limited to, a sample tray 302, microcentrifuges 304, a refrigerated energy rich homogenate holder 306, pH meters and standards, weighing balance, a refrigerated centrifuge 310, homogenizer 308, test tubes, spatulas, glassware, pipettes, liquid nitrogen, storage and distribution equipment, and at least one low temperature bio-extraction apparatus.

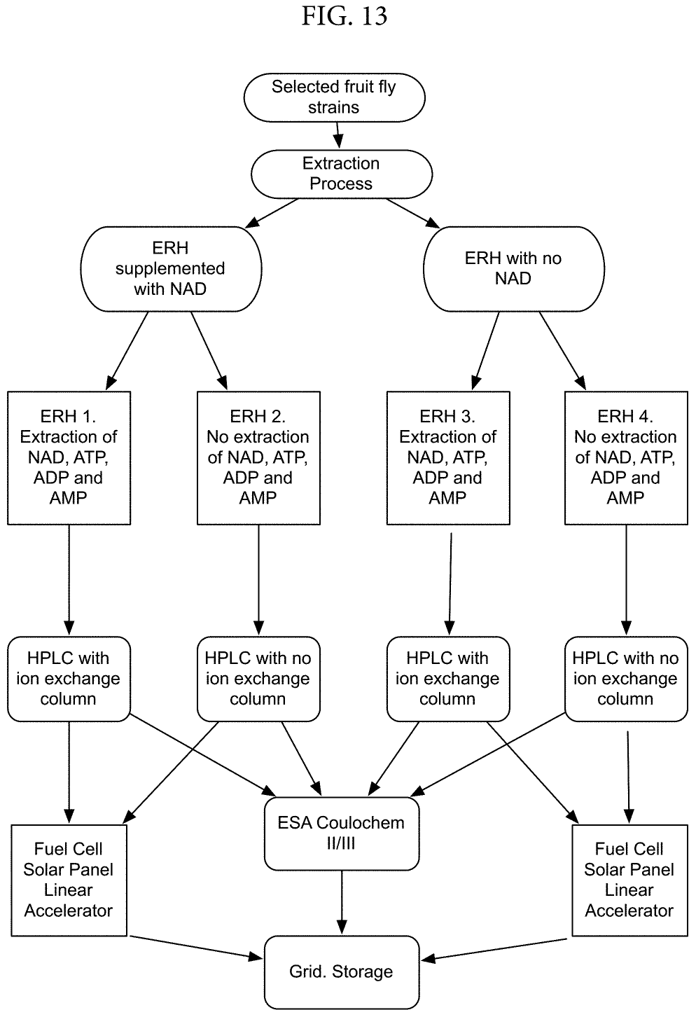

In general, the extraction process results in two forms of energy rich homogenates, untreated homogenate and homogenate treated with supplemental NAD, both of which can be further treated in two ways: no extraction of NAD, ATP, ADP, and AMP or extraction of NAD, ATP, ADP, and AMP using formic acid (for example, 4.2 M) and ammonium hydroxide (for example, 4.2 M) and freeze-thaw of homogenate. Therefore, as illustrated in FIG. 13, there are four energy rich homogenates available to enable assessment of the relative energy yield: (1) untreated with no extraction; (2) untreated with extraction; (3) treated with no extraction; and (4) treated with extraction.

More specifically, in one embodiment of the homogenate preparation portion of the extraction process, extraction may take place using single larval homogenates prepared from the third instar larvae of single cultures, as illustrated in FIG. 9. Larvae can be homogenized in pure water (for example, around 500 microL) at 0-3 C. Smaller portions of the homogenate, for example 100 microL portions, can be obtained from the initial combination and immediately transferred to centrifuge tubes on ice. Prior to addition of the homogenate to the tubes, the tubes may be empty or may be supplemented with, for example, distilled water or NAD. The amount of water or NAD in the tubes can vary, but in some embodiments, is 100 microL. The homogenate portion and the supplement, if any, can be mixed and stored on ice for a period of time (for example, 10 minutes) to facilitate metabolic activity.

In a second embodiment of the extraction process, extraction may take place using single larval homogenates prepared from the third instar larvae of single cultures. Larvae can be transferred to microcentrifuge tubes, weighed, and homogenized in predetermined amounts of ice-cold pure water (for example, 250 microL). NAD or pure water can be added to the microcentrifuge tubes in predetermined amounts or concentrations (for example, 250 microL of 0.01 M NAD or pure water). Alternatively, nothing can be added to the microcentrifuge tubes. The solutions can then be mixed and stored on ice for a period of time (for example, 40 minutes) to facilitate metabolic activity.

In some embodiments, the energy rich homogenate from the tubes described above in either embodiment can be immediately transferred to an assay/electron transfer system (for example, an ESA Coulochem II/III), as described below and illustrated in FIG. 13, or can be used in fuel cells, solar panels, or linear accelerators, as illustrated in FIG. 13.

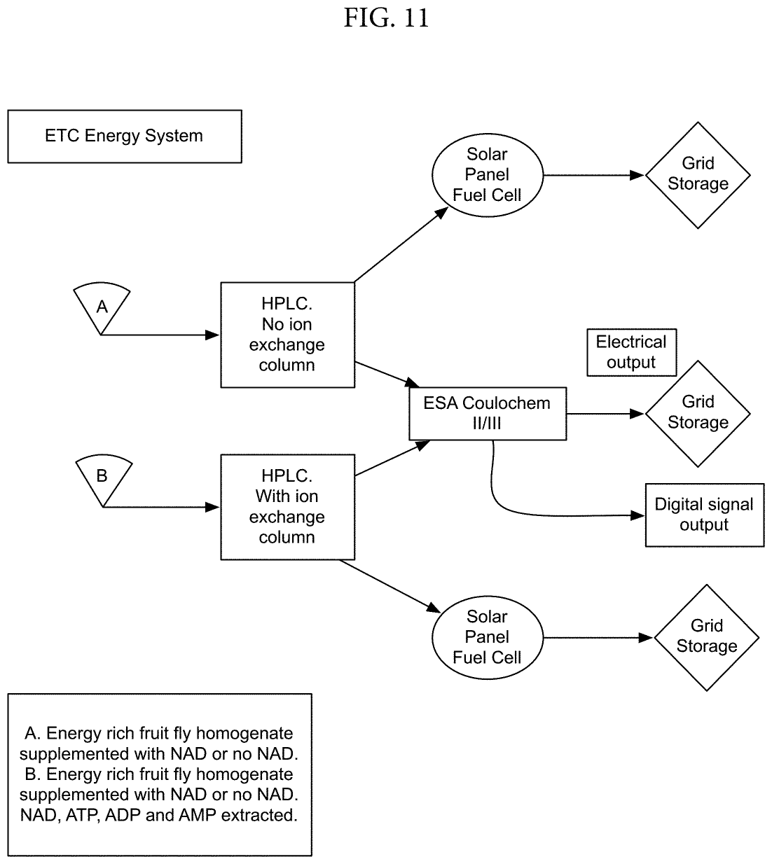

In another embodiment, NAD, ATP, ADP, or AMP can be extracted from the energy rich homogenate in the tubes described above by using formic acid and ammonium hydroxide (for example, 4.2 M formic acid and 4.2 M ammonium hydroxide). In this embodiment, metabolic activity is stopped in the energy rich homogenate. Following that treatment, the remaining energy rich homogenate can then be transferred to an assay/electron transfer system, as illustrated in FIGS. 10-11 and 13, or can be used in fuel cells, solar panels, or linear accelerators as energy for human use, as illustrated in FIGS. 11 and 13.

After the energy rich homogenate has been successfully created (i.e., homogenization of selected strains is complete), the supplemental NAD that can be added to the homogenized solution may not be used up. Additional use for the supplemental NAD is described in more detail below.

ETC Energy System: Transfer Process

In the transfer process, using the transfer system 200 illustrated in FIG. 2, the energy rich homogenate can be directly transferred to an electrochemical/coulometric instrument/detector, such as an ESA Coulochem II or III (abbreviated in the FIGS as CII or CIII) via a high-pressure liquid chromatography (HPLC) pump 208 or via a full HPLC set-up 206 and the output voltage can be assessed. A full HPLC set up 206 can include an HPLC pump 208, location for homogenate samples 210, separation column 212, mobile phase equipment 204, gradient creator, PDA detector 202, tubing 402, voltmeter 216, and a computing system.

The HPLC pump 208 or, alternatively, the full HPLC set up 206 can be connected to the coulometric instrument (for example, the coulometric device 214), which can couple the biologically-determined, enhanced ETC activity, a chemiosmotic process, to a complex electrochemical process in order to help transfer the energy (i.e., electrons and protons) from the fruit fly to the grid or to a storage device for human use. The energy rich homogenates can be directly transferred to the coulometric device 214 either via an HPLC pump 208 or via the full HPLC set up 206. The output voltage can then be assessed with the voltmeter 216 and the specific energy molecules collected for subsequent use.

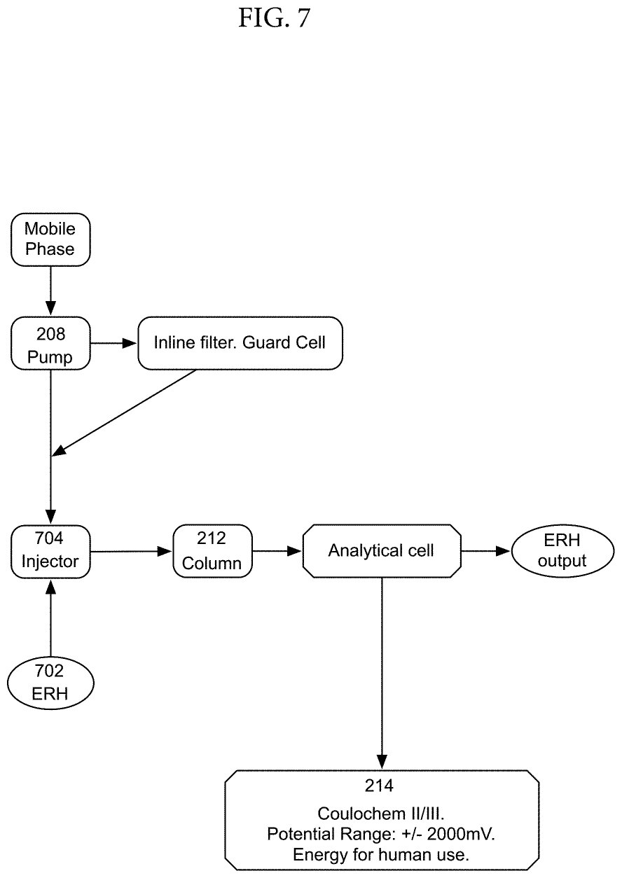

As illustrated in FIG. 7, after the HPLC pump 208 is connected to the coulometric device 214, the homogenate 702 can be injected manually with an injection valve 704. In one embodiment, a flow rate of 1 ml/min is used. In one embodiment, the HPLC pump 208 can be thoroughly primed and can deliver a consistent flow rate. If a separation column 212 is used, the separation column 212 can create backpressure so the HPLC pump 208 can function efficiently. Alternatively, a long piece of narrow bore tubing can create a backpressure coil. In another embodiment, a full HPLC set-up 206, which is fully automated for sample injections, could be used to transfer the homogenate to a coulometric device 214.

Fuel Cells

The energy rich homogenate can be used directly in a fuel cell as a novel reactive matrix. In one embodiment, a set-up similar to an enzymatic biofuel cell, or a modified enzymatic biofuel cell, is used that includes an anode and a cathode. The anode of the enzymatic biofuel cell can be catalyzed by oxidases suitable for conversion of bio-fuels or can be catalyzed by a complex of such enzymes for a complete oxidation of bio-fuels. For example, bio-fuel to be oxidized can be glucose, and the catalysts can include the fuel oxidizing enzymes glucose oxidase, glucose dehydrogenase and alcohol dehydrogenase. The cathode of the enzymatic biofuel cell can include an oxidoreductase that uses molecular oxygen as the ultimate electron acceptor and catalyzes reduction to water in neutral or slightly acidic media.

In the context of the modified enzymatic fuel cell, the energy rich homogenate, as described above, can act as the bio-fuel and NAD can be used as a catalyst to replace currently used enzymes. Alternatively, instead of fully replacing current fuels and enzymes, the energy rich homogenates can supplement the fuel and NAD can supplement the enzymes. A third option can include the use of oxygen as the substrate, NAD as the catalyst, and the energy rich homogenates as the source of electrons. The anode and electrode can further be separated by the proton exchange membrane (PEM).

Solar Panels

The energy rich homogenate can also be used directly in a solar panel as a novel reactive matrix. For example, in one embodiment, the disclosed system can couple to a solar voltaic cell. Standard solar voltaic cells include two silicon semiconductors between metal contacts, protected by a grid. Therefore, a solar voltaic cell can be integrated with the disclosed system by coupling the energy rich homogenate to the junction between the semiconductors. When, as described above, NAD is added to the energy rich homogenate, the NAD pool in the energy rich homogenate becomes relatively oxidized. Therefore, electron transport is enhanced from the oxidized NAD pool. The enhanced electron transfer can modify the redox potential of the solar voltaic cell causing accelerated electron transport between the two silicon semiconductors.

Linear Accelerators

The energy rich homogenate can also be used directly with components of a linear accelerator that collect the particles that are generated. Linear accelerators are, essentially, large electromagnets. Therefore, when the energy rich homogenates are combined with the linear accelerator components, electrons and protons may be readily transferred from the energy rich homogenates to storage devices or the grid. In sum, this process can extract electrons and protons from the energy rich homogenates by reversing the direction of flow through the system as it is normally used.

Storage Device

The bio-energy (ex: NAD, ATP, ADP, and AMP) and the electrical energy (electrons and protons) from the energy enhanced fruit fly strains are stored primarily in those selected fruit fly strains, similar to how energy from fossil fuels is stored in fossil fuels. The bio-energy and the electrical energy from the energy enhanced fruit fly strains can be released during the extraction and transfer process. More specifically, to directly and immediately use the energy stored in the fruit flies, bio-energy, electrons and protons can be extracted from the energy rich homogenates via a fuel cell, solar panel, linear accelerator, or ETC energy system, illustrated in FIGS. 10-11, and that electrical energy can be immediately used. However, instead of extracting the electrical energy from the fruit flies and immediately using the energy, the energy can be stored in, for example, capacitors, additional fuel cells, power plants, solar panels, or other systems.

In one example of fuel cell technology to be used as energy storage, the energy is derived from the fruit flies, as described above, and stored in the form of hydrogen. For example, excess electrical energy from the fruit flies can be fed into an electrolyser to split water into its constituent parts, oxygen and hydrogen. The hydrogen can then be stored in any type of fuel cell, which operates as the most efficient means of converting hydrogen back to electricity. Further, electrolysers and fuel cells are complementary technologies. Therefore, when energy is needed, the fuel cell can release the stored energy back to the grid. Alternatively, instead of releasing energy to the grid, the stored hydrogen can be diverted for sale to fuel cell electric vehicle owners, who use proton exchange membrane fuel cells to power their vehicles.

In one example of energy storage, the energy is derived from the fruit flies, as described above, converted to hydrogen, and stored in quinone-based flow batteries. In another example, the energy derived from the fruit flies can be converted into heat and the heat can be captured in thermal storage banks. One example of a thermal storage bank is where the converted thermal energy from the fruit flies is stored in molten salt, which can absorb extremely high temperatures without changing state.

Overview of the Photosynthesis Matrix

As mentioned above, this disclosure describes three system. The first, the bio-energy production system, has been described in detail above. The second, the photosynthesis matrix, is described below as a standalone system and as the third system: a combination of the first and second systems.

The disclosed photosynthesis matrix can be used to reduce atmospheric carbon dioxide levels by improving the naturally-occurring photosynthesis capabilities of plant material to absorb carbon dioxide and produce glucose, oxygen, and water. When used on a large scale, the photosynthesis matrix can improve greenhouse gas levels by accelerating rates of carbon dioxide reduction and, therefore, ameliorating environmental damage that has occurred due to global climate change.

A model system may be comprised of a sealed chamber, carbon dioxide, homogenized plant material (for example, a chloroplast solution comprised of spinach leaves or Chlamydomonas Reinhardtii), and an ATP solution. More specifically, in the basic model system, the homogenized plant material can be comprised of a chloroplast solution, which can be created and transferred to a tray. The tray can then be transferred into a sealed chamber where it can be positioned on top of a shaker. Carbon dioxide may be introduced into the chamber, and the gas concentration within the chamber can be measured over time using an ADI gas analyzer.

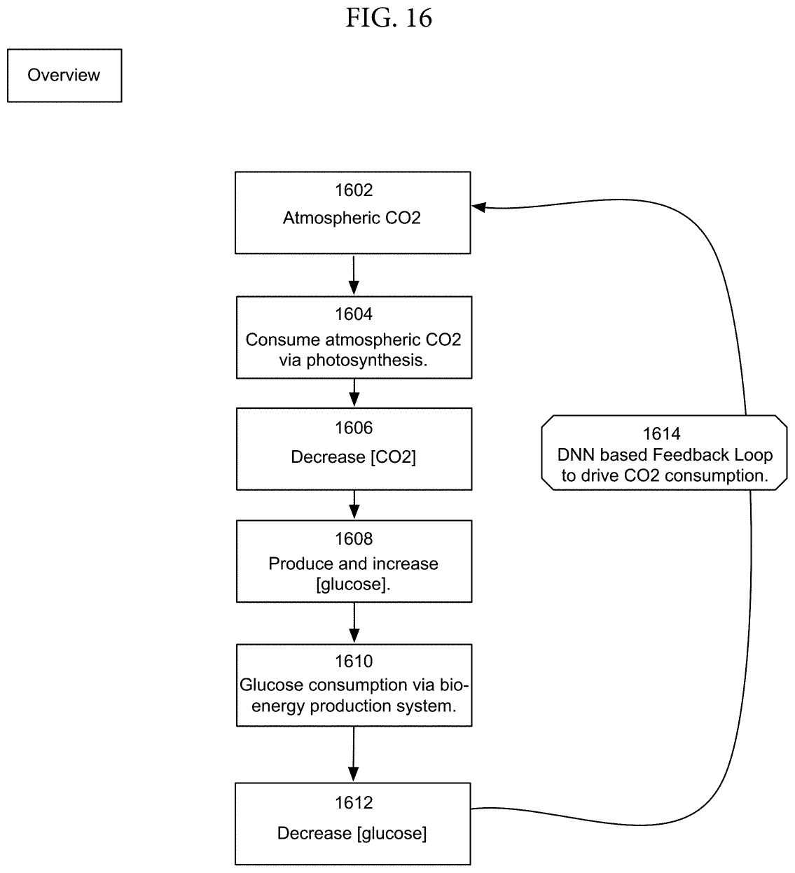

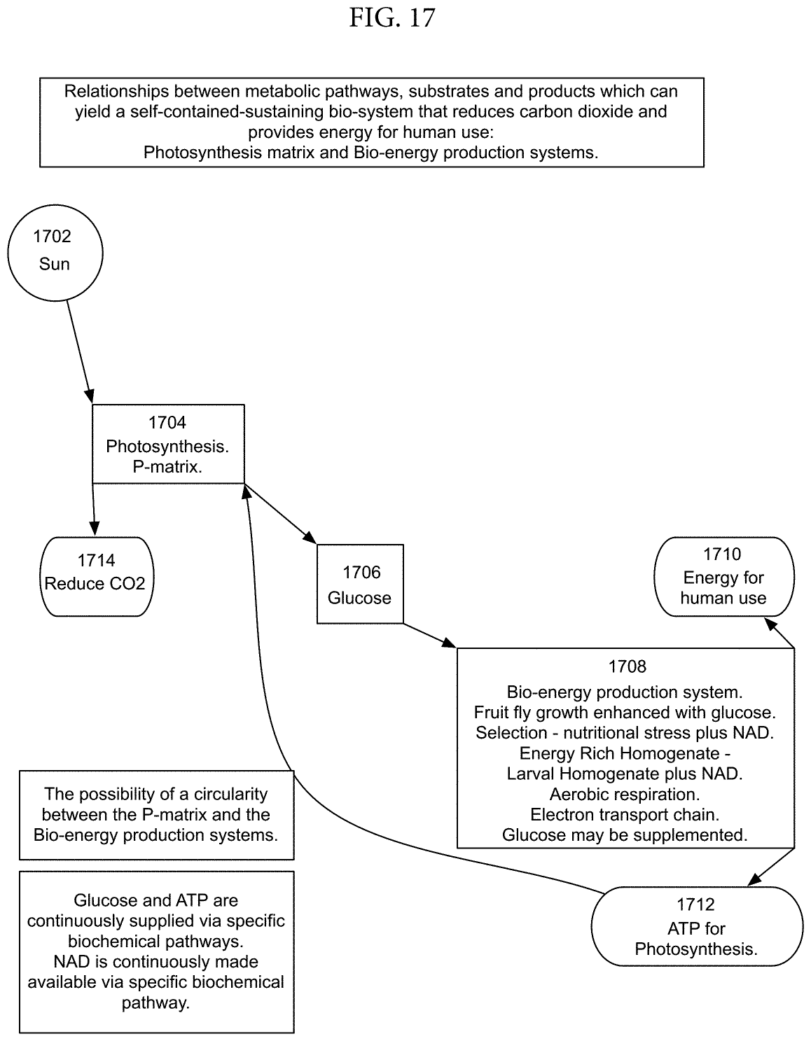

In some embodiments, due to the role of ATP and NADP as reducing equivalents in photosynthesis, ATP and/or NADP can be introduced into the chloroplast solution to increase photosynthetic activity and, therefore, expedite the process of reducing carbon dioxide concentrations. For example, carbon dioxide and water utilize ATP to create a glucose end product. In some cases, the ATP can be added as a pure ATP solution. In other cases, the ATP can be derived from the bio-energy production system to create a bio-feedback loop, as illustrated in FIGS. 16-17. FIG. 16 illustrates a brief overview of the outcomes of a combined system wherein atmospheric carbon dioxide 1602, when added to the system, is consumed via photosynthesis 1604, and the atmospheric concentration of carbon dioxide therefore decreases 1606. Photosynthesis also increases the concentration of glucose 1608, and the additional glucose can then be consumed by the bio-energy production system 1610. This decrease in glucose concentration 1612 causes a DNN-based feedback loop 1614, which drives the system to continue.

FIG. 17 illustrates another simplified version of this loop, wherein the photosynthesis matrix 1704, when provided with sunlight 1702, produces glucose 1706 that can be incorporated into the bio-energy production system 1708. The bio-energy production system 1708 then takes the glucose 1706 and uses it in the electron transport chain to produce energy for human use 1710 as well as additional ATP 1712. The additional ATP 1712 can be fed back to the photosynthesis matrix 1704 to expedite the process of reducing carbon dioxide concentrations 1714.

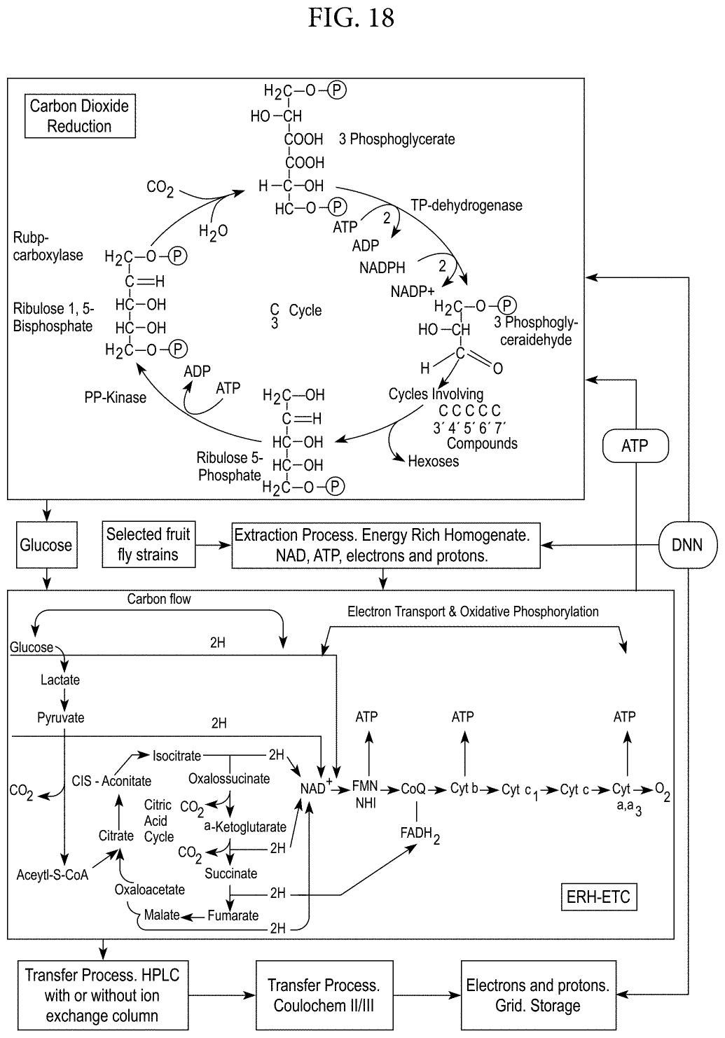

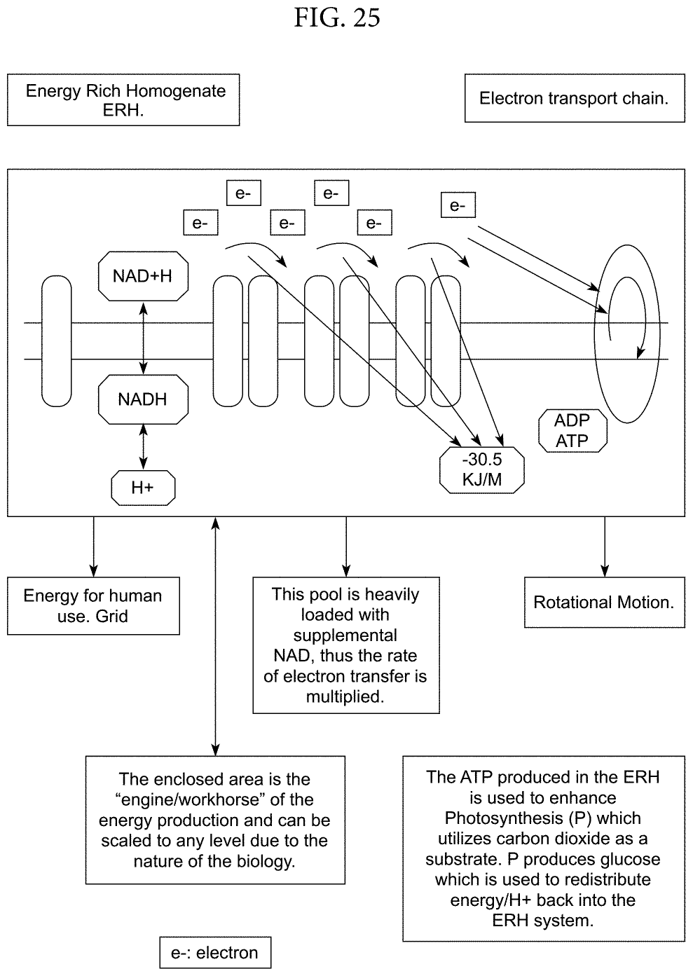

FIGS. 18 and 25 illustrate the more detailed chemical process of photosynthesis when combined with the ATP production of the bio-energy production system. After the energy rich homogenate has been successfully created (i.e., homogenization of selected strains is complete), the supplemental NAD that can be added to the homogenized solution may not be used up. For example, in FIGS. 18 and 25 the pool of NAD is, in theory, not degraded and NAD molecules can be repeatably metabolized (reduced) forming NADH and then metabolized (oxidized), and the electrons that are contained can be shuttled down the electron transport chain. This allows for continuous transfer of electrons in the form of hydrogen and, therefore, causes continual energy gathering. Further, as illustrated in FIG. 25, the supplemental NAD can enhance the NAD pool in the relevant pathways, leading to an increased rate of electron transfer and a multiplier effect in terms of energy availability. As noted, the ATP produced in the energy rich homogenate can be used to enhance the photosynthesis matrix, which uses carbon dioxide as a substrate. The photosynthesis matrix produces glucose and reduced carbon, which can be used to redistribute energy back into the energy rich homogenate or for other biochemical processes.

Therefore, disclosed herein are three embodiments of the photosynthesis matrix. The first is a chloroplast solution on its own. The second is a system wherein ATP is introduced to the chloroplast solution. The third is a system wherein the NAD-supplemented energy rich homogenate, as described above, is added to the chloroplast solution in order to add the ATP that will improve photosynthetic activity.

### Examples

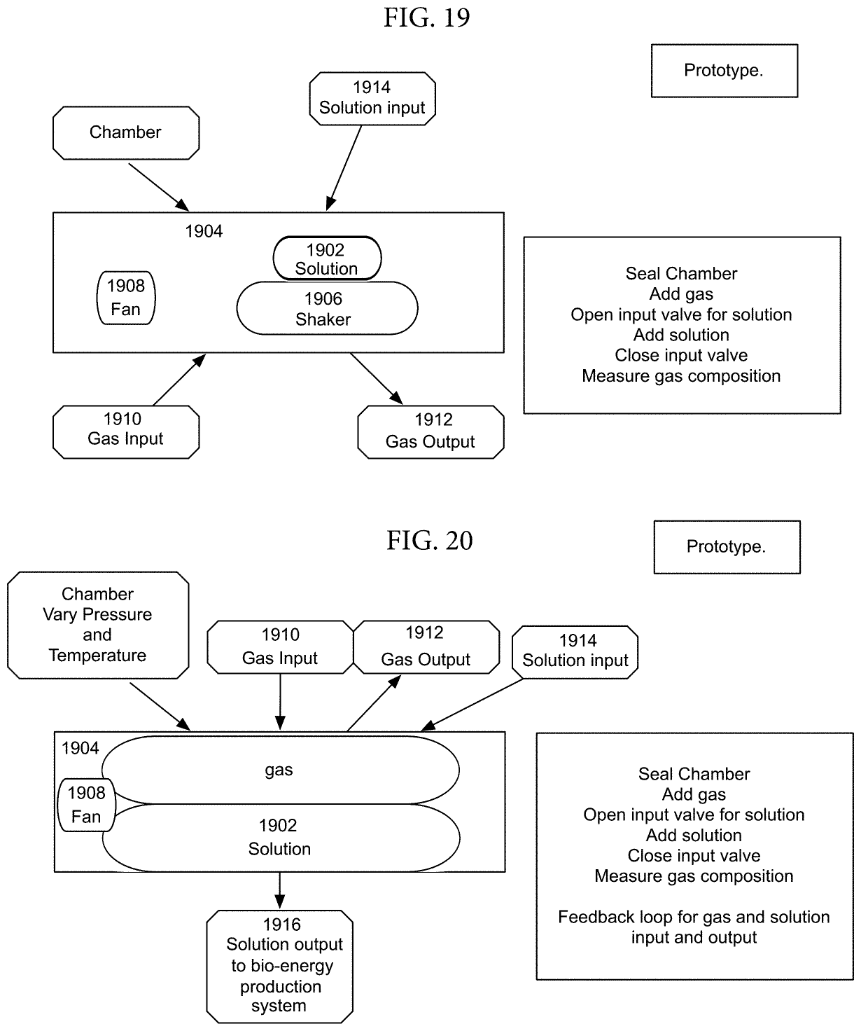

In experimental examples, as illustrated in FIGS. 19-20, a chloroplast solution 1902 was placed in a chamber 1904 on top of a shaker 1906. A fan 1908 was incorporated into the chamber 1904 to move the carbon dioxide molecules and put them in contact with the solution 1902, as would naturally occur in a real-world application of this system. To better control levels of carbon dioxide concentration in the chamber 1904, a gas input 1910 and output 1912 were constructed to lead directly into the chamber 1904. The solution input 1914 can also be built directly into the chamber 1904 to prevent the need for a “stabilization process” for the carbon dioxide. FIG. 20 includes an additional solution output 1916 where the chloroplast solution 1902 and its corresponding increased glucose levels can be removed and provided to the bio-energy production system described above.

In some embodiments, the homogenized plant material (i.e., the chloroplast solution) can be created by placing a tray of water under light for a predetermined amount of time (for example, overnight or for 24 hours), placing plant material (such as spinach leaves) on the surface of the water for a second predetermined amount of time (for example, one to three hours), homogenizing the plant material in 250 milliliters of ice cold 0.5M sucrose, and sieving the suspension (for example, through a funnel and one or more layers of cheesecloth into a 500 ml flask). To homogenize the material, it can be placed in a blender and lightly packed. It can then be filtered through the cheesecloth into the empty flask. In some cases, once filtered, 40 ml of cold 0.01M ATP may be added to the flask, mixed for two minutes with the plant extract, and then the entire solution transferred to the chamber. Alternatively, the solution can be transferred to the chamber without the addition of ATP. This procedure is scalable up or down.

In some embodiments, the solution can be introduced into the chamber through a front panel in the chamber. However, because this puts the solution and the chamber in contact with atmospheric carbon dioxide, results may be difficult to obtain. Therefore, in other embodiments, the solution can be introduced through a tube that runs through a small hole in a top panel of the chamber. In this embodiment, the solution can be introduced without interrupting the current carbon dioxide levels in the chamber and, therefore, it is possible to obtain accurate results sooner after the solution is introduced. To enable the gas to equilibrate better prior to introduction of the solution, the fans can be turned on, the empty tray placed on a shaker that has been activated, and the chamber sealed for a predetermined amount of time (for example, 5 minutes). Carbon dioxide can then be introduced into the chamber using the gas input, the chamber can be resealed, and a second predetermined time period can expire (for example, a second set of 5 minutes) to allow for the carbon dioxide to settle.

Once the carbon dioxide levels are set, the solution can then be introduced via the tube and added to the tray on top of the shaker using a solution input, which prevents the gas concentration in the chamber from fluctuating. After being added to the tray and the chamber resealed a third time, the solution can be mixed for two minutes. After mixing, the ADI gas analyzer can track changes in carbon dioxide concentration over time.

In additional experiments, the carbon dioxide concentration may be decreased significantly further by the addition of 40 milliliters 0.01M ATP, 220.4 mg ATP powder, or larval extract combined with ATP, NAD and sucrose. More specifically, the homogenized plant material supplemented with 40 milliliters 0.01M ATP can significantly decrease carbon dioxide concentrations compared to (i) the absence of a chloroplast solution, (ii) a chloroplast solution without any supplements, (iii) a chloroplast solution supplemented with 20 milliliters 0.01M NADP, and (iv) non-homogenized plant material in 250 milliliters of solution. Therefore, the experiments conducted support the conclusion that addition of ATP improves the ability of a chloroplast solution to decrease carbon dioxide concentrations.

Since ATP improves photosynthetic activity, an embodiment of a model system can incorporate the energy-enhanced biological organisms described above and the corresponding bioenergy production system. More specifically, the fruit fly strains selected for in the above-described bio-energy system (i.e., the energy rich homogenate combined with ATP, NAD and glucose) can act as primary sources of energy storage, and when the fruit fly diet is supplemented with NAD, the proportion of ATP available via the fruit fly homogenate, as well as the ATP/ADP ratio, increases, as illustrated in FIG. 8.

In one embodiment, 3200 milligrams of larvae extract can be homogenized in 40 milliliters of water. This solution can be sieved and 40 milliliters 0.01M NAD can be added. After seven minutes, the entire solution can be frozen (with the option also to freeze dry). It can then be thawed, sieved, and added to the chloroplast suspension.

In another embodiment, 40 mg third instar larvae can be homogenized in 0.5 ml cold water with a subsequent addition of 0.01M NAD. Thus, for 40 ml of supplement as used in a plant-extract with ATP, 3.2 grams of third instar larvae could be used. Thus, larval mass can first be determined and then the volume of water for mixing can be calculated. In one example, if there were 3200 mg of third instar larval mass, then 40 ml water could be mixed in. If 2000 mg of third instar larval mass was created, then 25 ml water could be mixed in.

More specifically, to create a chamber solution, the following steps can be taken: collect and weigh third instar larvae, calculate the volume of cold water to add, homogenize the larvae in the calculated amount of cold water, sieve the larval homogenate, add 0.01 M chilled NAD to facilitate electron transport chain activity and ATP production, allow the solution to react for a predetermined amount of time (for example, 10 minutes), then add the solution to the flask with the chloroplast solution, mix for two minutes, and transfer to the test chamber. In some embodiments, the solution may then be centrifuged to separate it into supernatant and pellet components. In other embodiments, the solution may separate into supernatant and pellet without the need for centrifuging. Each of the components (supernatant and pellet) can be added separately to plant extracts, mixed, and then introduced into the chamber after chamber equilibrium.

In some embodiments, further steps can be taken. For example, the pH and conductivity levels of the combined solution can be matched to the pH levels and conductivity of the plant extract on its own; the larval homogenate can be freeze-dried prior to addition to the plant extract; temperature and pressure changes can be made to maximize the desired outcome (i.e., removal of carbon dioxide); and/or ATP can be extracted from the energy rich homogenate using HPLC procedures and used as the ATP source for the photosynthesis matrix.

Application

The model system can be adapted to real-world applications and is flexible enough to be scaled as needed. More specifically, the photosynthesis matrix can be scaled up or down to accommodate the desired carbon dioxide reduction rate at a specific point in time. Therefore, as the amount of carbon dioxide continues to increase, the photosynthesis matrix can be scaled up to consume the excess carbon dioxide and to prevent it from remaining in the atmosphere.

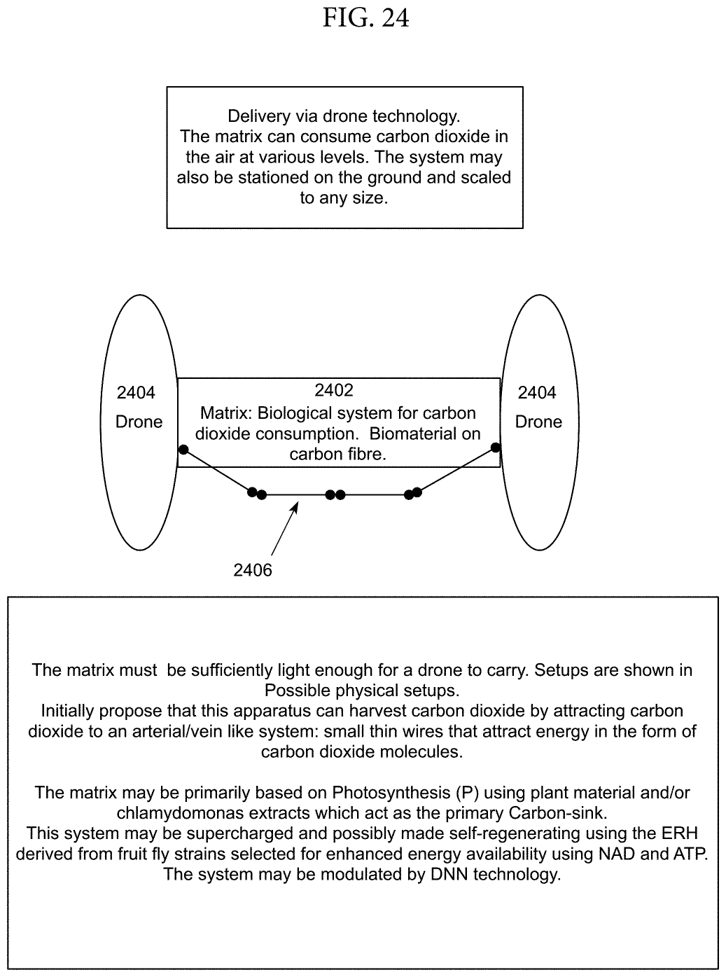

One example of a scaled system includes delivery of the chloroplast solution via drone technology, as illustrated in FIG. 24. More specifically, the photosynthetic matrix 2402, which includes the desired chloroplast solution, can consume carbon dioxide in the air at various elevations by being positioned on a drone or between two or more drones 2404. The drone apparatus can attract carbon dioxide to an arterial/vein-like system 2406 comprised of small, thin wires that attract energy in the form of carbon dioxide molecules. As the carbon dioxide molecules are attracted to the device, the chloroplast solution can use them to drive photosynthesis and create glucose, oxygen, and water.

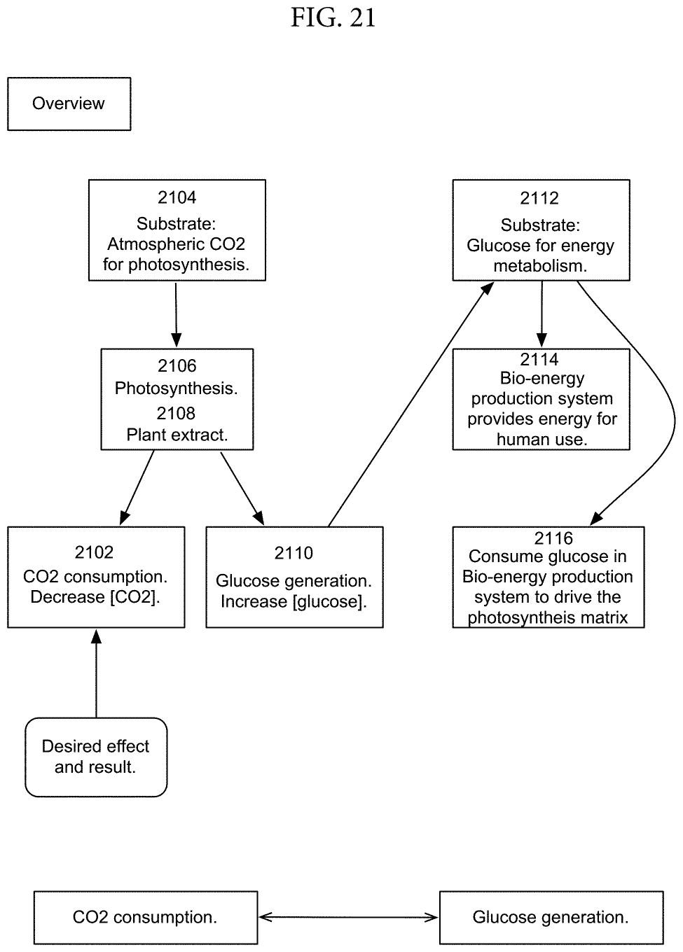

As illustrated in FIGS. 16 and 21, the glucose provided by the photosynthesis matrix can, in turn, be used to feed the bio-energy production system. The bio-energy production system can then, in turn, produce biological organisms with increased levels of ATP and increased ratios of ATP/ADP. These organisms can be processed as described above to create an energy rich homogenate with increased levels of ATP and increased ratios of ATP/ADP that can be used in the chloroplast solution and the photosynthesis matrix to enable the matrix to continue to absorb carbon dioxide at an expedited rate. When the energy rich homogenate is successfully coupled to the photosynthesis matrix by way of specific reactants and products, the solution can repetitively cycle through the appropriate biochemical pathways. More specifically, a self-regenerating perpetuating bioenergy system can be created by providing recyclable key molecules for the energy rich homogenate system and the photosynthesis matrix, as illustrated in FIG. 18.

As illustrated in FIG. 21, a first step for the system may be carbon dioxide consumption 2102. More specifically, atmospheric carbon dioxide (the substrate 2104), photosynthesis (the mechanism 2106), plant extract 2108 (the matrix) and sunlight as an energy source can collectively consume carbon dioxide 2102 and create glucose 2110, the substrate 2112 for a second step, which is energy metabolism. In this second step, use of the energy rich homogenate system described above produces bio-energy, electrons, protons, and water for human use 2114. Consumption of glucose 2116 can regulate the photosynthesis matrix activity as well as carbon dioxide levels. Further, ATP can be generated for use in the photosynthesis matrix.

In one embodiment, an enhanced procedure for generating larval material to be used with the photosynthesis matrix can include the following steps: (1) adults mated and allowed to egg-lay for three days at 20 C; (2) adults removed and cultures yeasted over 4-6 days, wherein the yeast solution can be 1.8 g/60 ml with 5 ml added to bottle cultures and 4 drops to vial cultures; (3) cultures transferred to 18 C and monitored until larvae are available; (4) larvae collected from cultures using yeast solution as an extraction solution; and (5) transfer of the yeast solution that contains the larvae to a filter paper for final larval collection.

As mentioned above, glucose provided by the photosynthesis matrix can power the bio-energy production system (for example, glucose can be provided during the selection process), and the ATP produced through the bio-energy production system can be used and/or stored for future human use, as illustrated in FIG. 21. More specifically, energy can be sent back and forth between the two systems at a constant rate (i.e., the energy is recycled via a feedback loop) and, in the process, carbon dioxide can be consumed. As the energy rich homogenate consumes the glucose created by the photosynthesis matrix, it can provide electrons and protons for human use.

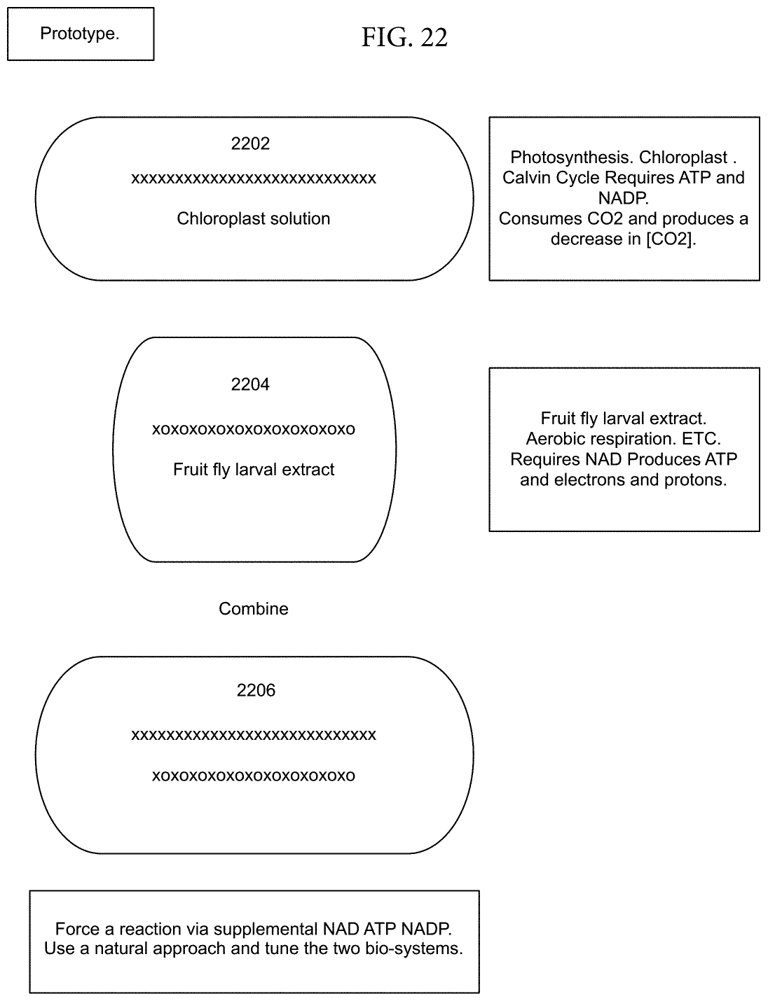

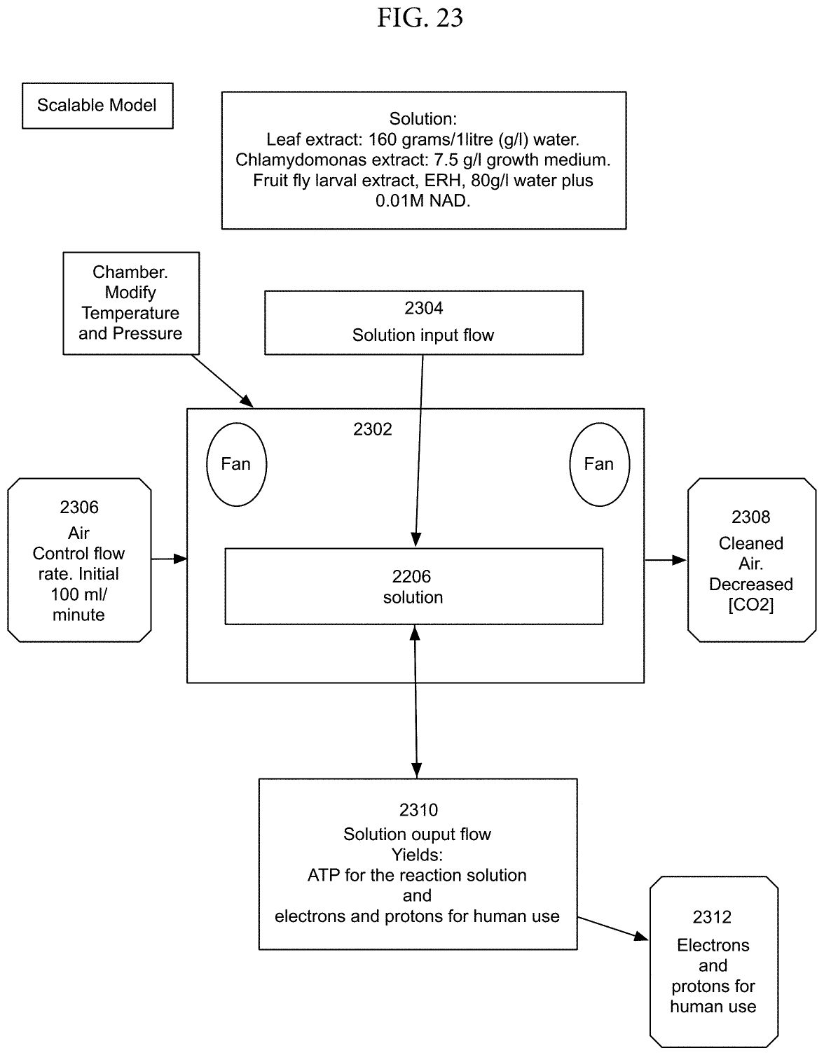

In some embodiments, a combined solution can be utilized wherein the photosynthesis matrix and the bio-energy production system are mixed together, as illustrated in FIGS. 22-23. In a prototype model, illustrated in FIG. 22, the chloroplast solution 2202 and the energy rich homogenate 2204 are combined together into a combined solution 2206. In a scalable model, as illustrated in FIG. 23, the combined solution 2206 can be added to a chamber 2302 via a solution input 2304, as described above, and the solution 2206 can include plant extract (160 grams/1 liter water), Chlamydomonas extract (7.5 grams/liter growth medium), and/or the energy rich homogenate (80 grams/1 liter water plus 0.01M NAD). The temperature and pressure in the chamber 2302 can be modified and the flow rate of air into the chamber 2302 can be controlled (for example 100 milliliter/minute). The system can then remove carbon dioxide from air that is input into the chamber 2306 and output the cleaned air from the chamber 2308. Further, the solution that goes into the chamber can be removed 2310, and it may include additional ATP for the reaction solution as well as electrons and protons for human use 2312.

This combined solution can be self-sustaining and useful in real world applications. For example, if a drone system is used to put the interconnected photosynthesis matrix and bio-energy production systems in contact with carbon dioxide, both systems could operate together to reduce or eliminate time needed to transfer ATP and glucose between the bio-energy production system and the photosynthesis matrix. As illustrated in FIG. 24 and mentioned above, the drone matrix may be comprised of a biological system for carbon dioxide consumption and can include biomaterial on carbon fiber. This matrix may be primarily based on photosynthesis using plant material and/or Chlamydomonas extracts, which can act as the primary carbon-sink. The system may also be improved and self-generating by using the energy rich homogenate, and it may be modulated by deep neural network technology.

Other real-world embodiments are envisioned. For example, to drive photosynthesis more effectively in a whole plant organism, in vivo uptake of key biomolecules may be designed into the system. More specifically, a powder substance that disperses biomolecules via nanoliposome technology may further actively drive photosynthetic activity. This powder substance can be nutrient rich, can contain necessary bioactive reagents/compounds/molecules, and can enhance bio-energy metabolism. To recreate the above-described improved photosynthetic effect in a whole plant organism, the powder substance can contain necessary bioactive reagents and vesicular transport technology to collapse the improved photosynthetic effect and recreate it in the whole plant organism. Outputs from the system, such as the removed carbon dioxide that is stored in the photosynthetic material and sugars (for ex: glucose), can be used for future biochemical processes.

The most effective method to deliver the nanoliposome powder may include any of the following options: (1) photosynthesis matrix: add the powder directly to the chloroplast solution; (2) whole plant organism: deliver the powder as a direct supplement to the soil of growing plants so that the powder (which may be released by nanoliposomes) interacts directly with the plants' roots, allowing the plants to uptake the necessary molecules (ATP) via their root systems to drive an increased rate of photosynthesis, which might decrease the growing period, increase carbon dioxide reduction, or improve plant size; (3) a combination of approaches 1 and 2.

The formula for this powder can be created using, for example, crystallization, freeze-drying, lyophilization, electrophoresis, and high-performance liquid chromatography. One example of the powder may include the photosynthesis solution with the addition of commercial ATP. A second example of the powder may include the photosynthesis solution with the addition of the energy rich homogenate supplemented with NAD. In this case, aerobic respiration and photosynthesis are placed together. The ATP from the second example can be extracted from the homogenate using HPLC or it can be directly delivered from the homogenate.

Cellular Arrays

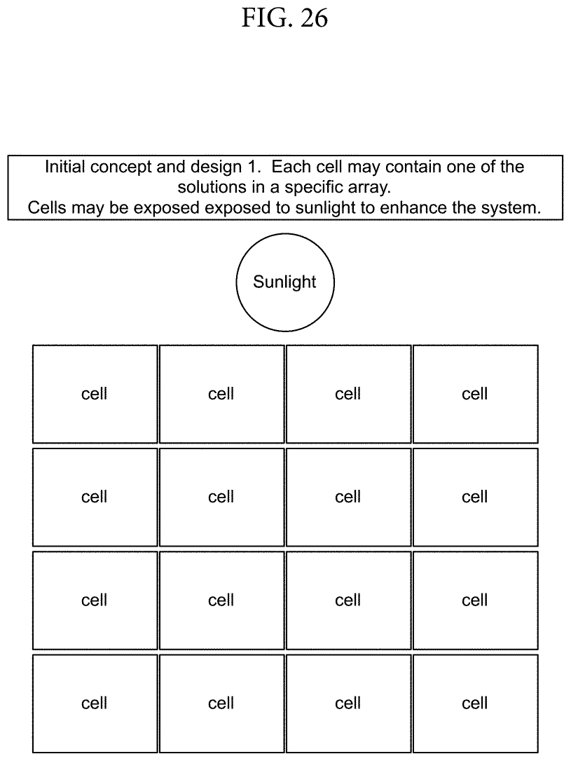

As illustrated in FIGS. 26-32, individual or arrays of cells may be used to implement the above-described energy rich homogenate environment and/or photosynthesis matrix. The various physical setups can range from very small to very large. For example, the setup can be mounted on a drone for local, precise, low-level operations, as described above. The setup is also envisioned to scale up so it can function and operate at the level of domestic houses, office complexes, apartments, factories, and/or other large buildings, similar to solar panels. In this manner, the system can ultimately replace the current fossil fuel dependent power plants.

In each embodiment, the solution in the cell may be an energy rich homogenate solution (ERH), a photosynthesis matrix solution (P), or a combined solution (C) that includes the energy rich homogenate supplemented with NAD and photosynthesis matrix. The interface technology may be based on standard HPLC and Coulochem II/III techniques. In some cases, HPLC and Coulochem II/III cells may be enhanced with Linear Accelerator technology.

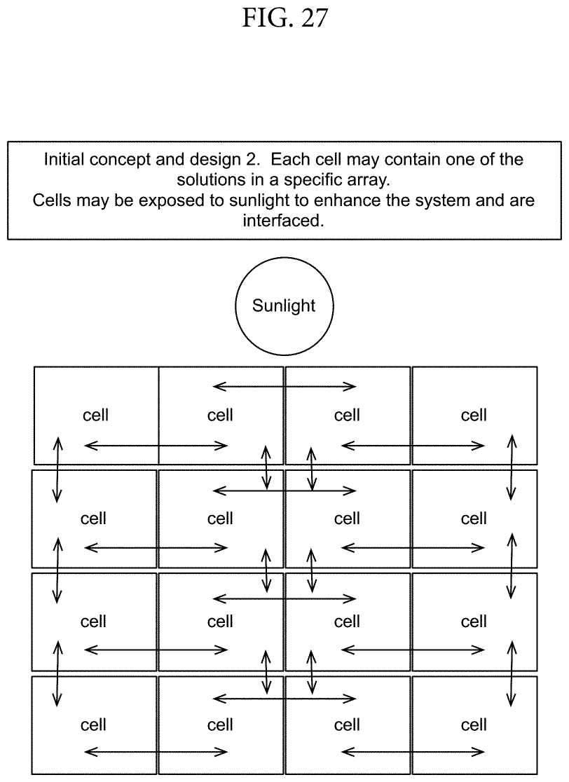

FIGS. 26-27 illustrate the initial concept and design of cells within an array. FIG. 26 is considered to be a basic layout of cells within an array that additional concepts can develop upon, as described below. Each cell in FIG. 26 may contain one or both of the ERH and photosynthesis solutions, and the cells may be exposed to sunlight to enhance the system. FIG. 27 illustrates the next stage, which incorporates connection and interface between the cells within the array, wherein the cells and the interface between the cells are focal points.

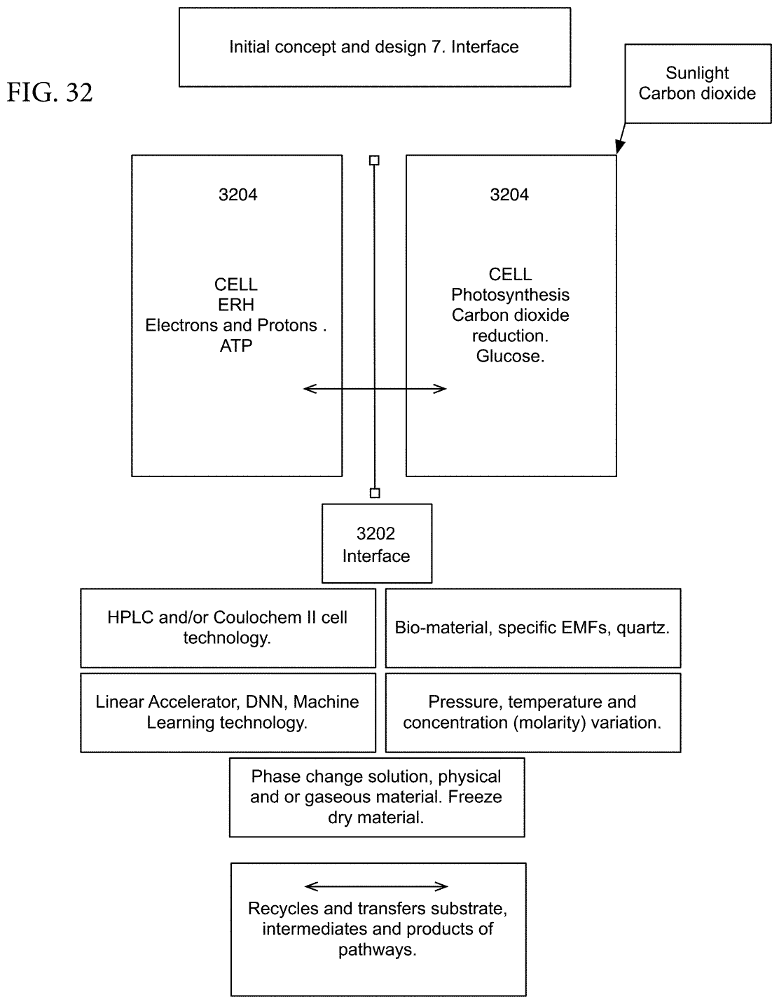

As with FIG. 26, in the array illustrated in FIG. 27 each cell may contain one or both of the solutions, and those cells may be exposed to sunlight to enhance the system. The array may drive itself, such that it can be structured and configured to enable each cell to interface with each neighboring cell, as illustrated in FIG. 32. Interfacing allows for a passive transfer and exchange of products such as, but not limited to, bio-material or bio-solutions, and this transfer and exchange can facilitate metabolic activity, decrease carbon dioxide concentrations, and create energy. In some embodiments, the interface 3202 between two cells 3204 can be a barrier that allows movement of products across it, as illustrated in FIG. 32. For example, the barrier may be a porous barrier that allows for products of a certain size to constantly pass through. Alternatively, the barrier may be a solid barrier that can be opened to periodically allow all products to pass through.

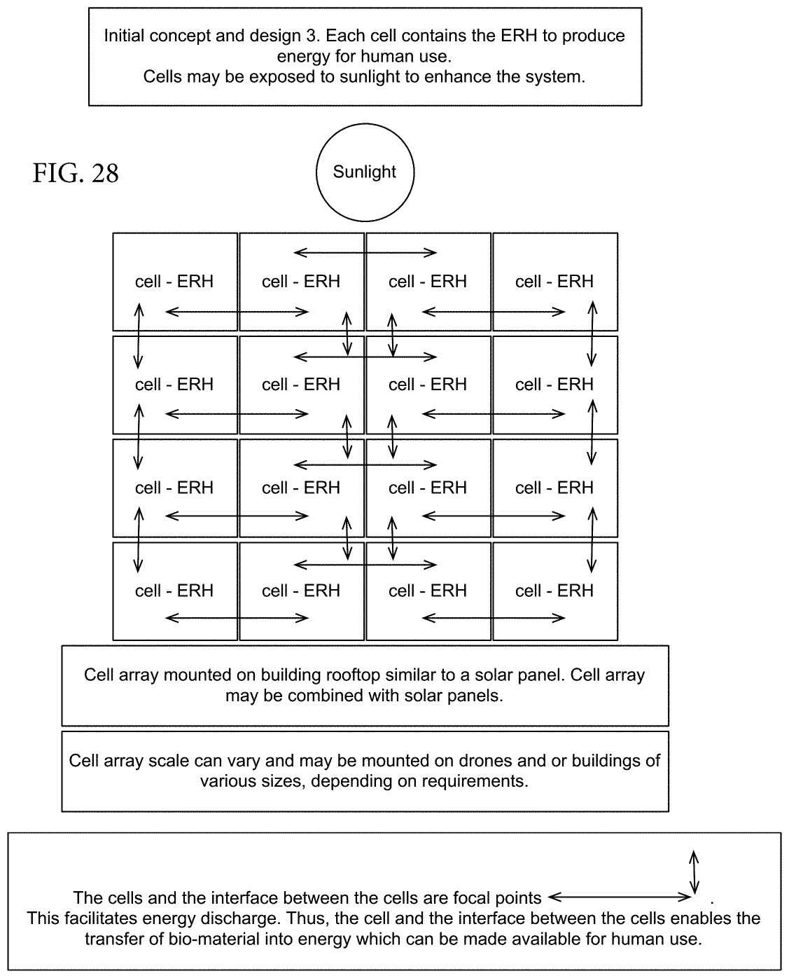

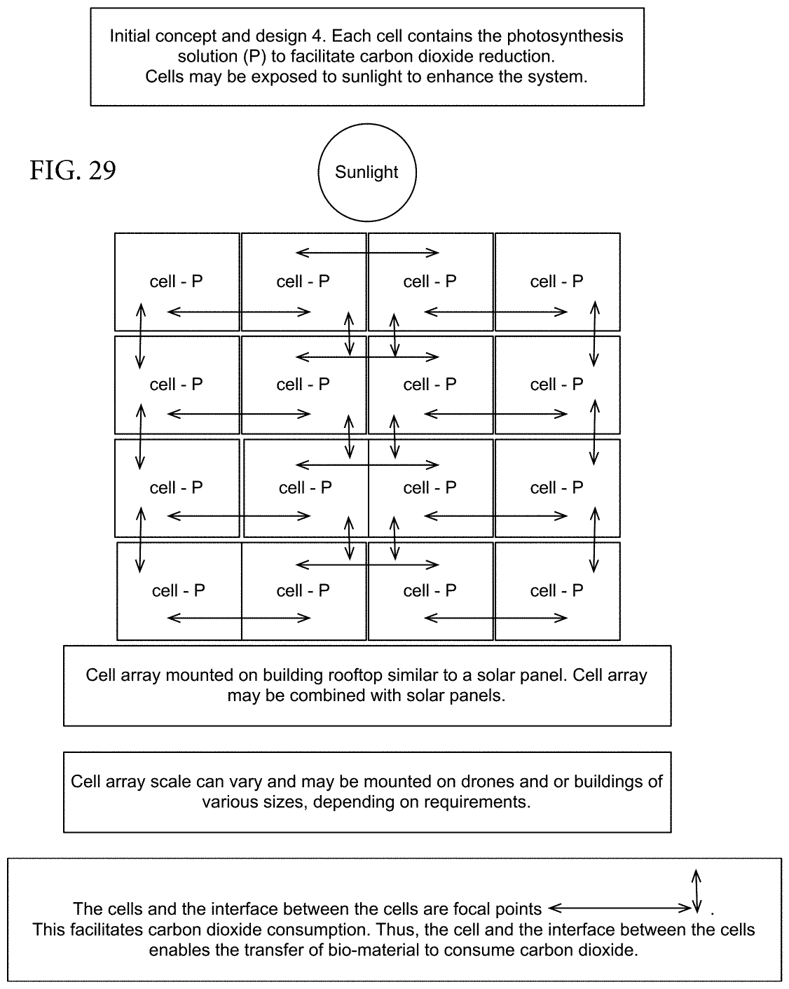

As described above, an energy rich homogenate input to the cell may result in an output of electrons and protons for ATP and human use. Additionally, a photosynthesis matrix input to the cell that includes carbon dioxide and the photosynthesis solution may result in an output of glucose as well as air/gas with a decreased concentration of carbon dioxide. Since sunlight is less beneficial in an energy rich homogenate-only solution, illustrated in FIG. 28 with only ERH cells, setups aimed at decreasing carbon dioxide concentrations may include cells that contain the photosynthesis solution, as illustrated in FIG. 29. This photosynthesis solution can facilitate carbon dioxide reduction, and sunlight can be used to enhance its effectiveness.

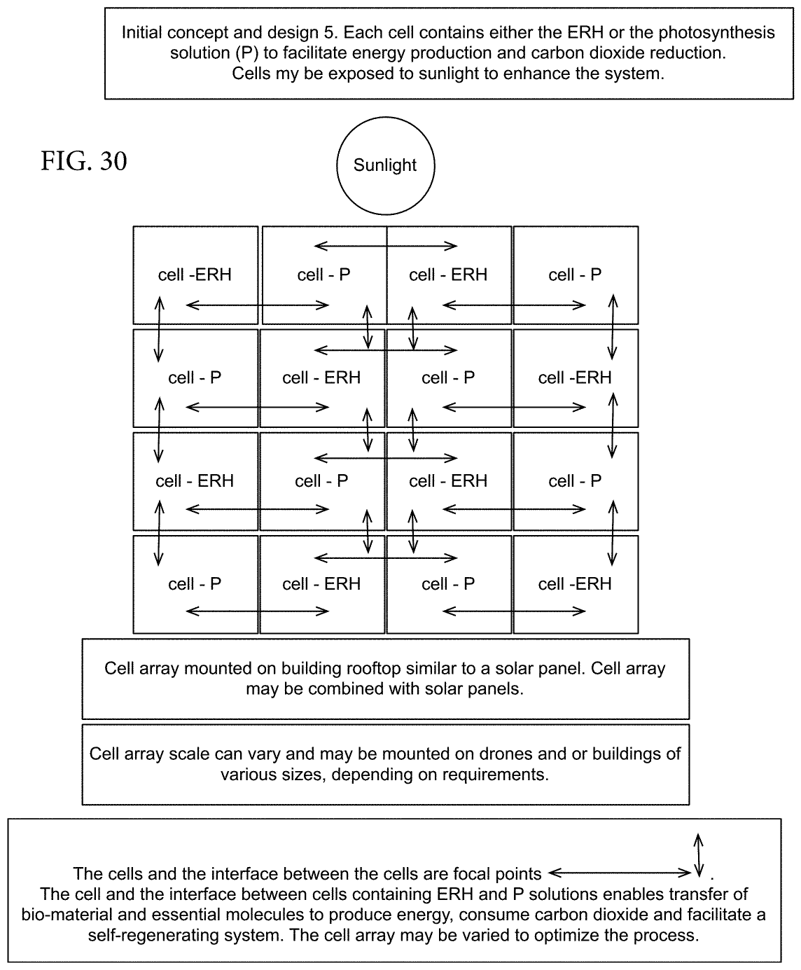

As FIG. 30 illustrates, in some embodiments, each cell may include the energy rich homogenate solution (ERH cell) or the photosynthesis matrix solution (P cell). However, since each cell can connect to, and interface with, each of its adjacent cells, the outputs from each cell can include certain elements that enable the array to self-regulate and remain self-sustaining. These elements can include ATP for P cells and glucose for the ERH cells. Additionally, other elements for human benefit such as electrons and protons for energy use as well as air/gas with a decreased concentration of carbon dioxide may be output from the ERH and P cells, respectively. To facilitate interfacing with opposite cells, each ERH cell can be surrounded on each internal side by a P cell and each P cell can be surrounded on each internal side by an ERH cell, as illustrated in FIG. 30. An example of an ERH cell and a P cell interfacing with each other is illustrated in FIG. 32. More specifically, FIG. 32 illustrates the interface between two cells, wherein one cell has the energy rich homogenate solution and the other cell has the photosynthesis solution. As shown, the interface enables substrates, intermediates, and products of each of the pathways to recycle and transfer between each other.

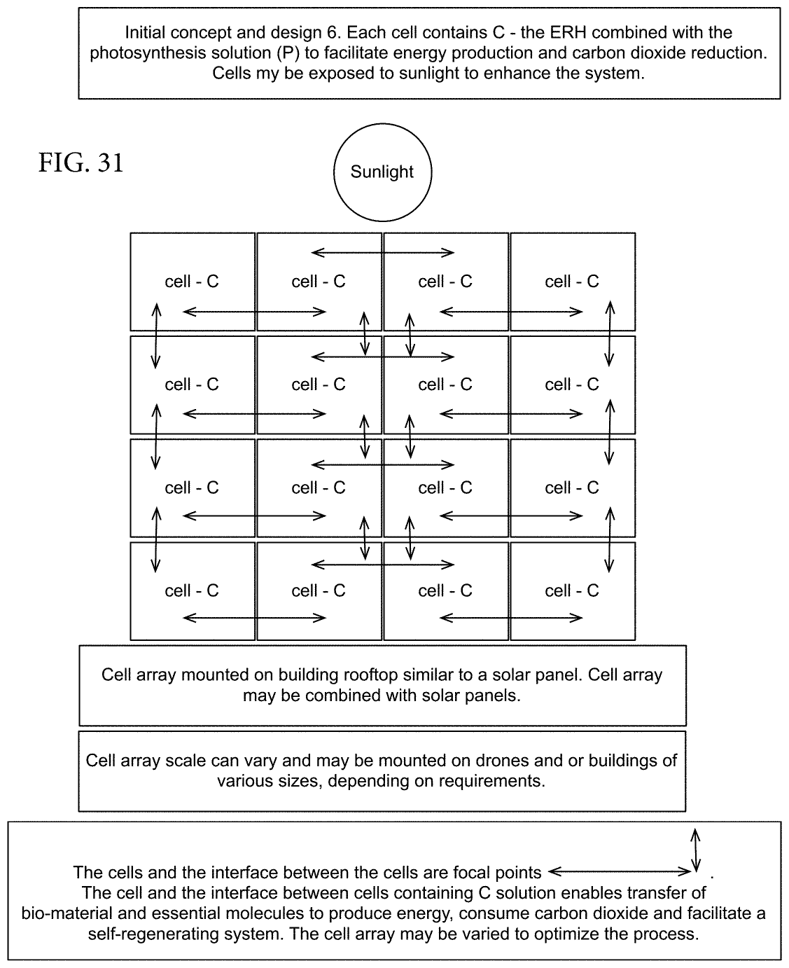

In some embodiments, instead of each cell being an ERH cell or a P cell, each cell in the array may include a combined solution of the energy rich homogenate and the photosynthesis matrix, as illustrated in FIG. 31. More specifically, the setup illustrated in FIG. 31 allows each of the cells to interface with adjacent cells and to transfer essential molecules. In addition to allowing cells to interface with each other, the setup can allow the transfer of substrates, intermediates, and products of pathways both between adjacent cells and within the cells themselves. This transfer process can help to facilitate a self-regenerating system. To further optimize this process, the cellular array may be varied from what is illustrated herein.

In some embodiments, the cells can be arranged independently from each other. The cells can be in the form of a single panel or a three-dimensional structure. By creating a cellular array, the surface area is maximized and creates ideal energy yield and carbon dioxide reduction. The cell size can be very small, which can further maximize the energy yield and carbon dioxide reduction. Ultimately, it may be possible to establish the cell based upon a single electron transport chain pathway and/or a single photosynthesis pathway.

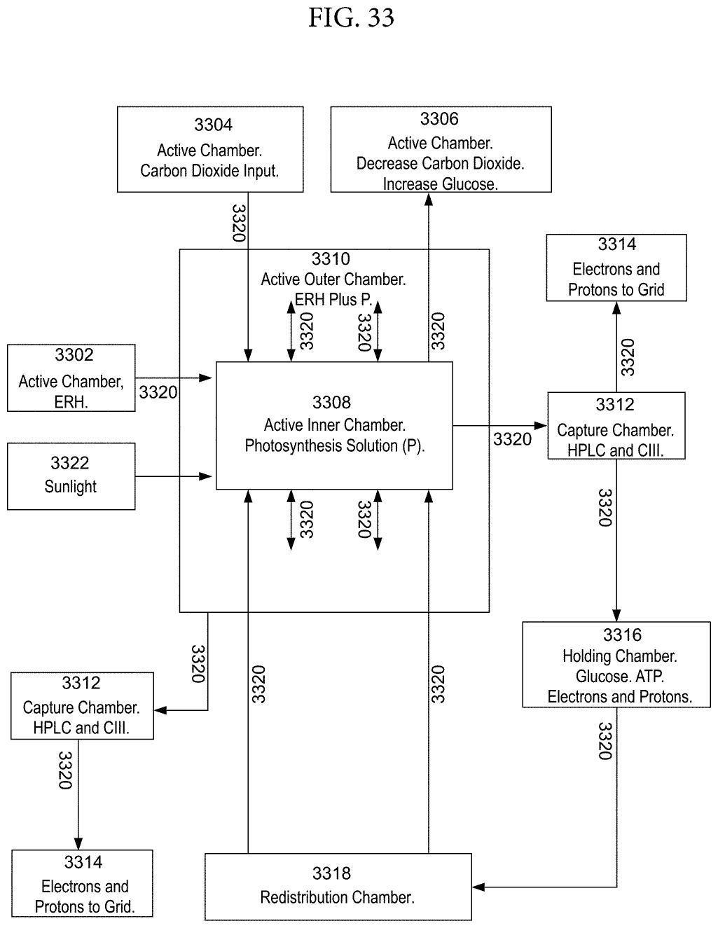

While FIGS. 26-32 illustrate a cellular array that is more passive in nature, FIGS. 33-35 illustrate the functional aspects of chambers within arrays that are more active in nature. The active nature of the arrays illustrated in FIGS. 33-35 enable application of the disclosed interfacing system at the level of county-wide areas or perhaps even larger, rather than the level of domestic houses, office complexes, apartments, factories, and/or other large buildings for the passive interfacing system illustrated in FIGS. 26-32. In some embodiments, the active arrays can operate similar to an HPLC in that each chamber can independently hold a solution separate from other chambers until a valve is activated. The valve, when opened, can allow the solution to be pulled out of the chamber by, for example, a solvent and transported to a new chamber through a tube or pipe. More specifically, the extract can be loaded into a tube system that can cycle through the material as needed and act as a filter. This tube system can be actively placed in high emission areas, attached to drone systems, or used across communities.

In some embodiments, it is envisioned that the HPLC hardware and technology will be used in the fabrication of the designs. This would include HPLC grade chambers, tubing and glassware, valves, standard injection loops, pumps, transducers, filters, and advanced software to regulate the transfers and control the reactions and the capture process. The constructs can be used to replace, on a large scale, the fossil fuel burning power plants and/or nuclear plants.

More specifically, FIGS. 33-35 illustrate the layouts and constructs of three advanced concepts and designs. The chambers used in these concepts and designs can be configured to actively hold and transfer the energy rich homogenate and photosynthesis matrix solutions to other specific chambers to facilitate biochemical reactions that yield products, such as desired molecules and compounds. These products can then be redistributed to other chambers to facilitate further reactions. Alternatively, or additionally, the products (for example, electrons, protons, ATP, water) can be accessed for human use. Here, the chambers are Active Chambers, Capture Chambers, Holding Chambers, Redistribution Chambers, and Clark Chambers.

The Active Chambers can contain the specific reactions of the energy rich homogenate and photosynthesis matrix. The Capture Chambers can capture the molecules and compounds for future use and can be based on the HPLC techniques for ATP, the Coulochem III (abbreviated as CIII in the Figures) techniques for electrons and protons, and other techniques for glucose, carbon dioxide and water. The Holding Chamber can hold molecules and compounds for future use and they can be based on required pathways, substrates, and cofactors. The Redistribution Chambers can hold sets of required molecules and compounds for movement to the other cells for recycling reactions. The Clark Chambers can be the ultimate design that reduces the distance between all of the components to maximize efficiency and sensitivity.

FIG. 33 illustrates one array layout incorporating the above-described chambers and valves 3320 that can hold and release solutions allowing them to flow from one chamber to another. The active input only chambers can contain the energy rich homogenate 3302, carbon dioxide 3304, or the photosynthesis matrix solution 3306, and the active input/output chambers can contain the photosynthesis matrix 3308 (for example, in an inner chamber) or the combined solution 3310 (for example, in an outer chamber) while also receiving sunlight 3322. The capture chambers 3312 can capture molecules from the input/output chambers and can continue outputting electrons and protons to an energy grid 3314. Alternatively, or additionally, the capture chambers 3312 may output products such as glucose, ATP, electrons and protons to the holding chamber 3316. The holding chamber can hold on to these products for a predetermined amount of time and can then release them to the redistribution chamber 3318, where they can be redistributed to other chambers earlier in the cycle (for example, the active input/output chambers 3308, 3310).

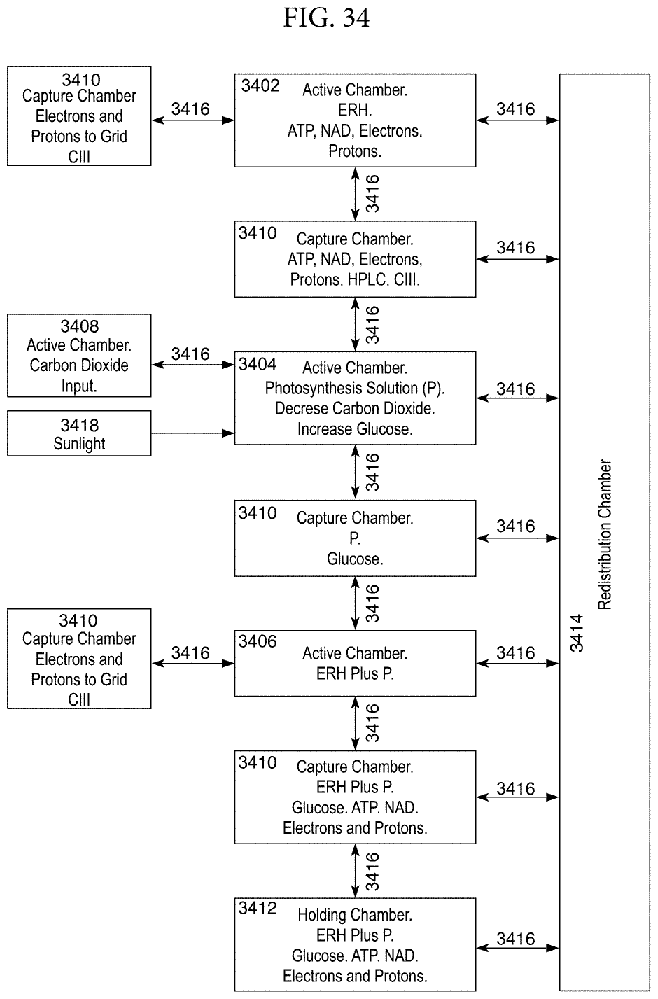

FIG. 34 illustrates a second array layout that operates similarly to the layout in FIG. 33, but wherein it has no inner or outer chambers layered on each other. Instead, it has three separate main active chambers wherein one contains the ERH 3402, one contains the photosynthesis solution 3404 and can receive sunlight 3418 in addition to products, and one contains the combined ERH and photosynthesis solution 3406. A fourth active chamber 3408 can input and output carbon dioxide to the photosynthesis chamber 3404. Each of these active chambers can output to one or more capture chambers 3410, wherein the capture chambers 3410 capture the output product specific to the contained solution in the active chambers 3402, 3404, 3406 (ATP, NAD, electrons/protons, and/or glucose). The holding chamber 3412, as with the first array, can hold all of these products. Another difference between the first and second array is that the second array allows for all chambers to have input and output valves 3416. Therefore, all of the chambers, with the exception of the active chamber 3408 and the capture chamber that can send electrons and protons to the grid, are able to input and output molecules and compounds with the redistribution chamber 3414. Therefore, almost every chamber can be in input and output communication with another chamber through the use of the redistribution chamber 3414.

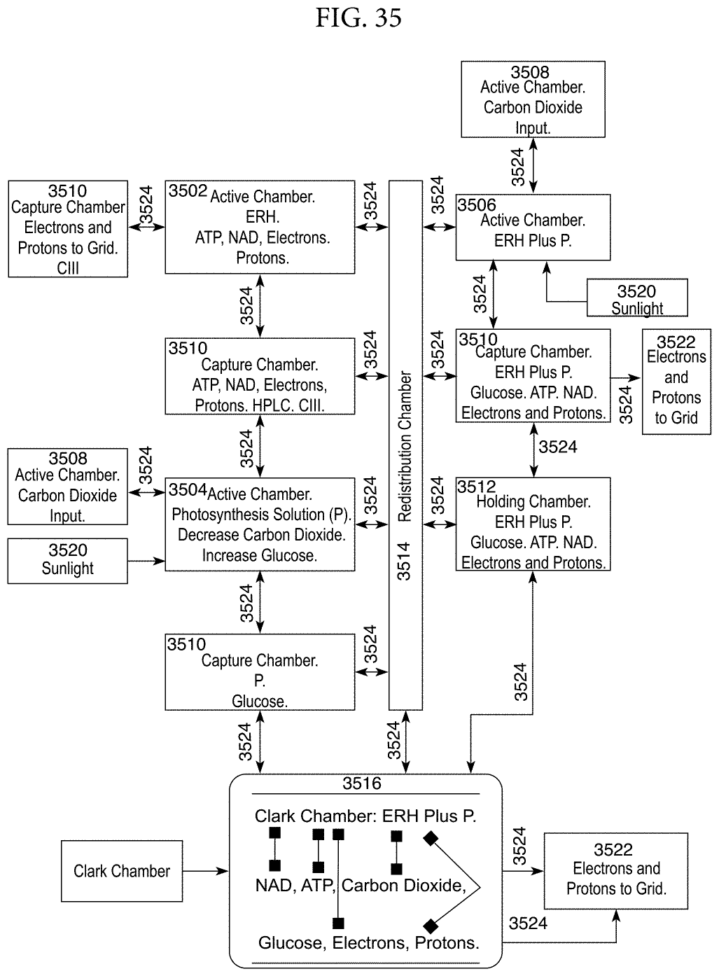

The third array layout is illustrated in FIG. 35 and, similar to FIGS. 33-34, incorporates active chambers that contain the ERH 3502, photosynthesis solution 3504, combined solution 3506, and carbon dioxide input 3508 that only interacts with active chamber 3504. Both chambers incorporating the photosynthesis solution 3504, 3506 can also incorporate a sunlight input 3520. The capture chambers 3510, as with above, can capture the output product specific to the contained solution in the active chambers 3502, 3504, 3506 (ATP, NAD, electrons/protons, and/or glucose). The holding chamber 3512 can collect and hold onto all of the products through a capture chamber 3510, the redistribution center 3514, or through the Clark chamber 3516. By incorporating the Clark chamber 3516, the system reduces the distance between all of the components to maximize efficiency and sensitivity. In this third array, it is envisioned that at least one capture chamber 3510 and the Clark chamber 3516 out electrons and protons to the grid 3518. As with the second array, the third array allows for all chambers to have input and output valves 3524.

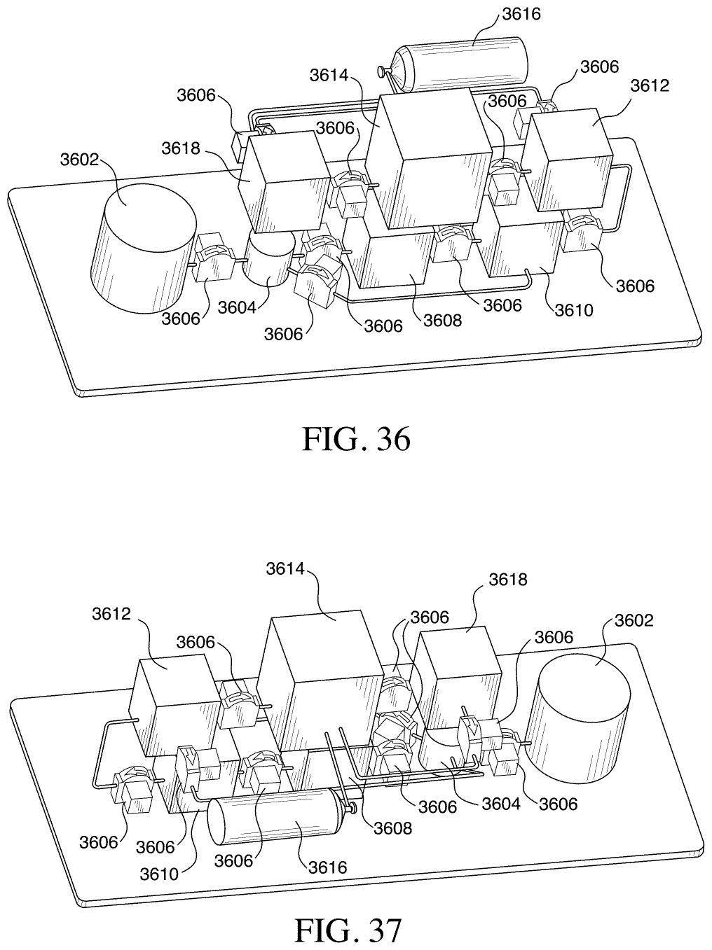

FIGS. 36-39 illustrate example assemblies of the above-described advanced concepts and designs, and FIGS. 40-41 illustrate interior details of the components. The example assemblies in FIGS. 36-39 can include components such as, but not limited to, photosynthesis chambers, energy rich homogenate chambers, biomolecule sensors, coulochem cells, filters (for example, CO2 filters), input reservoirs for the energy rich homogenate chamber, CO2 inputs, photosynthesis inputs, and pumps (for example, peristaltic pumps) connecting each of these components to each other. While there is overlap in the components included in each of these examples, the layouts can differ. FIG. 40 illustrates interior details of the energy rich homogenate chamber. FIG. 41 illustrates interior details of the photosynthesis chamber, which can function as a CO2 filter.

More specifically, FIGS. 36 and 37 illustrate an assembly having an input reservoir 3602 for the energy rich homogenate chamber 3604 with a pump 3606 moving material from the input reservoir 3602 to the energy rich homogenate chamber 3604. The pump 3606 may be, for example, a peristaltic pump. Material from the energy rich homogenate chamber 3604, details of which are illustrated in FIG. 40, can then be pumped through the coulochem cell 3608 and to a filter chamber 3610. Alternatively, material from the energy rich homogenate chamber 3604 can be pumped directly to the filter chamber 3610. From the filter chamber 3610, a pump 3606 can move the material to a biomolecule sensor 3612. The biomolecule sensor 3612 can have two output pumps 3606, a first that can move material from the biomolecule sensor 3612 to the photosynthesis chamber 3614 and a second that can circulate the material back to the energy rich homogenate chamber 3604. Material that gets pumped from the first biomolecule sensor 3612 to the photosynthesis chamber 3614, details of which are illustrated in FIG. 41, can be combined with CO2 from a CO2 input 3616 and then pumped to the second biomolecule sensor 3618. Material from the second biomolecule sensor 3618 can then be redirected and pumped back into the photosynthesis chamber 3614. In this example, the two biomolecule sensors 3612/3618, the photosynthesis chamber 3614, and the CO2 input 3616 can be placed above the input reservoir for the energy rich homogenate chamber 3602, the energy rich homogenate chamber 3604, the coulochem cell 3608, and the filter chamber 3610. Further, the CO2 input 3616 can be placed behind the photosynthesis chamber 3614.

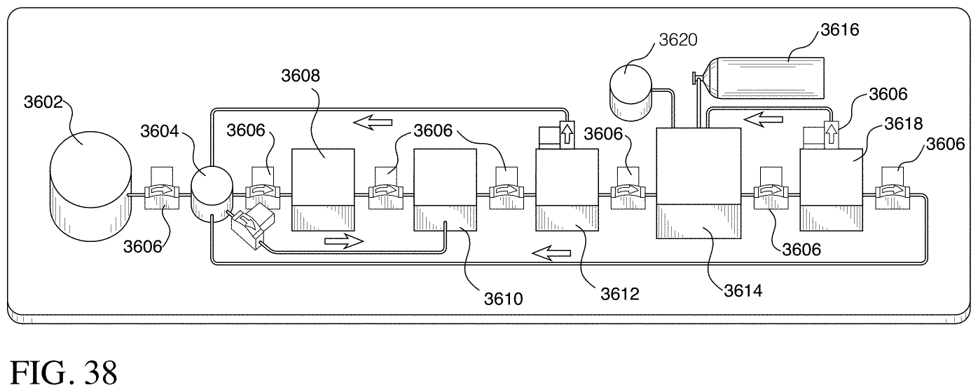

FIG. 38 illustrates a second assembly that is similar to the first and that has an input reservoir 3602 for the energy rich homogenate chamber 3604 with a pump 3606 moving material from the input reservoir 3602 to the energy rich homogenate chamber 3604. The pump 3606 may be, for example, a peristaltic pump. Material from the energy rich homogenate chamber 3604, details of which are illustrated in FIG. 40, can then be pumped through the coulochem cell 3608 and to a filter chamber 3610. Alternatively, material from the energy rich homogenate chamber 3604 can be pumped directly to the filter chamber 3610. From the filter chamber 3610, a pump 3606 can move the material to a biomolecule sensor 3612. The biomolecule sensor 3612 can have two output pumps 3606, a first that can move material from the biomolecule sensor 3612 to the photosynthesis chamber 3614 and a second that can circulate the material back to the energy rich homogenate chamber 3604. Material that gets pumped from the first biomolecule sensor 3612 to the photosynthesis chamber 3614, details of which are illustrated in FIG. 41, can be combined with CO2 from a CO2 input 3616 and photosynthesis material from a photosynthesis input 3620. That combined material can then be pumped to the second biomolecule sensor 3618. Material from the second biomolecule sensor 3618 can be circulated and pumped back into the photosynthesis chamber 3614 or can be pumped back to the energy rich homogenate chamber 3604. In this example, the two biomolecule sensors 3612/3618, the photosynthesis chamber 3614, and the CO2 input 3616 are all on the same plane as the input reservoir for the energy rich homogenate chamber 3602, the energy rich homogenate chamber 3604, the coulochem cell 3608, and the filter chamber 3610. More specifically, the input reservoir 3602, energy rich homogenate chamber 3604, coulochem cell 3608, filter chamber 3610, first biomolecule sensor 3612, photosynthesis chamber 3614, and second biomolecule sensor 3618 are in line, and the photosynthesis input 3620 and CO2 input 3616 are behind the photosynthesis chamber.

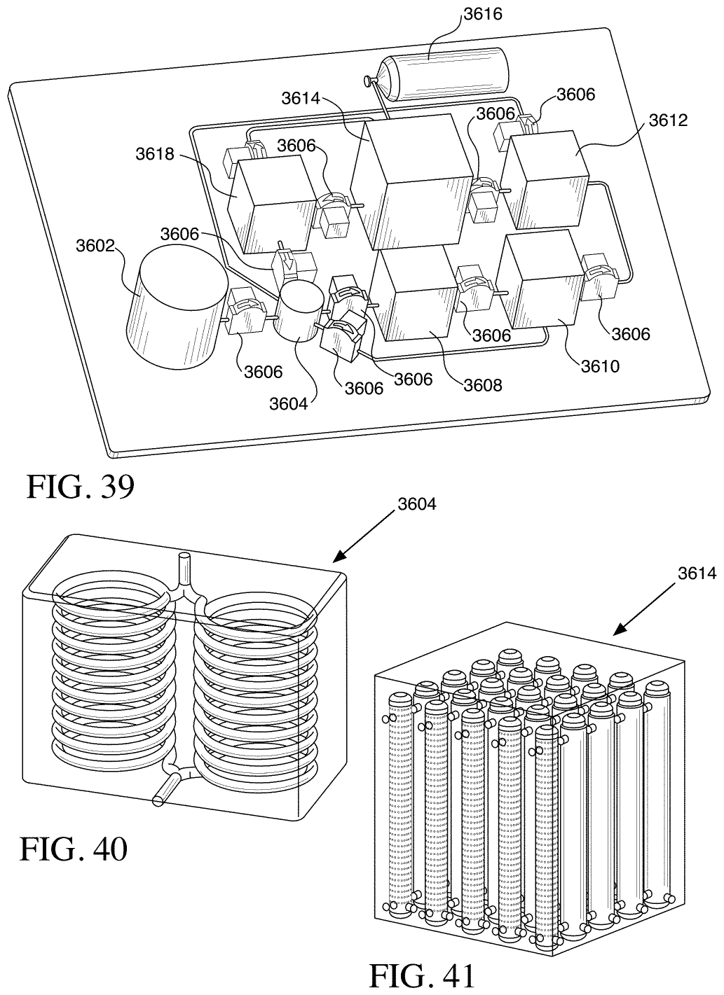

FIG. 39 illustrates a third assembly that is similar to the first and second assemblies. This third example assembly has an input reservoir 3602 for the energy rich homogenate chamber 3604 with a pump 3606 moving material from the input reservoir 3602 to the energy rich homogenate chamber 3604. The pump 3606 may be, for example, a peristaltic pump. Material from the energy rich homogenate chamber 3604, details of which are illustrated in FIG. 40, can then be pumped through the coulochem cell 3608 and to a filter chamber 3610. Alternatively, material from the energy rich homogenate chamber 3604 can be pumped directly to the filter chamber 3610. From the filter chamber 3610, a pump 3606 can move the material to a biomolecule sensor 3612. The biomolecule sensor 3612 can have two output pumps 3606, a first that can move material from the biomolecule sensor 3612 to the photosynthesis chamber 3614 and a second that can circulate the material back to the energy rich homogenate chamber 3604. Material that gets pumped from the first biomolecule sensor 3612 to the photosynthesis chamber 3614, details of which are illustrated in FIG. 41, can be combined with CO2 from a CO2 input 3616. That combined material can then be pumped to the second biomolecule sensor 3618. Material from the second biomolecule sensor 3618 can be circulated and pumped back into the photosynthesis chamber 3614 or can be pumped back to the energy rich homogenate chamber 3604. In this example, the two biomolecule sensors 3612/3618, the photosynthesis chamber 3614, and the CO2 input 3616 are all on the same plane as the input reservoir for the energy rich homogenate chamber 3602, the energy rich homogenate chamber 3604, the coulochem cell 3608, and the filter chamber 3610. However, the two biomolecule sensors 3612/3618 and the photosynthesis chamber 3614 are positioned in line with each other but out of line with the input reservoir 3602, energy rich homogenate chamber 3604, coulochem cell 3608, and filter chamber 3610, which are in line with each other. Similar to the embodiments in FIGS. 36-37 and 38, the CO2 input 3616 is behind the photosynthesis chamber 3614.

The photosynthesis chamber (i.e., CO2 filter), illustrated in FIG. 41, can be comprised of a perforated, elongate chamber that is covered by a clear tube, which allows free flow of material and light to penetrate the chloroplast and/or chloroplast solution. The photosynthetic extract and/or filtered material and CO2 can be loaded into an inferior end of the chamber while gas, glucose, and/or spent extract can be released from the superior end. Multiple chambers can be used together to maximize the effect. In some embodiments, the extract can be agitated within the chamber but in other embodiments, there is no agitation. To maintain continued bioactivity, there may be a continuous flow of the photosynthetic extract and/or agitation of the extract. Further, there may be passive and/or active CO2 uptake into the chamber.

The various embodiments described above are provided by way of illustration only and should not be construed to limit the claims attached hereto. Those skilled in the art will readily recognize various modifications and changes that may be made without following the example embodiments and applications illustrated and described herein and without departing from the true spirit and scope of the following claims.

1. An interconnected photosynthesis matrix and bio-energy production system, the interconnected system comprising:
a bio-energy production system comprising:
a selection process, wherein the selection process is applied to a first organism strain for a plurality of generations to create a second organism strain with enhanced energy availability; and
an extraction process, wherein the extraction process creates an energy rich homogenate from the energy enhanced organism strain; and
a photosynthesis matrix comprising:
carbon dioxide; and
a chloroplast solution having homogenized plant material and an ATP solution;
wherein the ATP solution is derived from the energy rich homogenate.

2. The system of claim 1, wherein the bio-energy production system creates electrons and protons for human use.

3. The system of claim 1, wherein the photosynthesis matrix consumes the carbon dioxide and produces glucose.

4. The system of claim 3, wherein the photosynthesis matrix is housed on at least one drone, the drone including wires that attract additional carbon dioxide molecules.

5. The system of claim 3, wherein the glucose from the photosynthesis matrix is used as a food source for the bio-energy production system.

6. The system of claim 5, wherein the ATP solution is used by the photosynthesis matrix at the same rate that the glucose is used by the bio-energy production system.

7. The system of claim 5, wherein the glucose is used by the bio-energy production system during the selection process.

8. The system of claim 5, wherein the interconnected system is incorporated into a passive cellular array.

9. The system of claim 8, wherein each cell in the passive cellular array includes both the energy rich homogenate solution and the chloroplast solution.

10. The system of claim 8, wherein each cell in the passive cellular array includes either the energy rich homogenate solution or the chloroplast solution.

11. The system of claim 10, wherein each energy rich homogenate solution cell is surrounded by chloroplast solution cells and each chloroplast solution cell is surrounded by energy rich homogenate solution cells.

12. The system of claim 11, wherein each cell interfaces with each adjacent cell to transfer output elements.

13. The system of claim 12, wherein the output elements include ATP from the energy rich homogenate solution cells and glucose from the chloroplast solution cells.

14. The system of claim 5, wherein the interconnected system is incorporated into an active array comprising:
a plurality of active chambers, each active chamber containing the energy rich homogenate, the chloroplast solution, or a combined solution having both;
a capture chamber that captures ATP, electrons, and protons from each chamber having the energy rich homogenate and glucose from each chamber having the chloroplast solution;
a holding chamber that accepts and holds ATP, electrons, protons, and glucose for future use; and
a redistribution chamber that accepts and redistributes ATP, electrons, protons, and glucose to other chambers within the active array.

15. The system of claim 14, wherein each cell in the active array further includes a Clark chamber that reduces the connection distance between each of the other chambers.

16. The system of claim 1, wherein the interconnected system is incorporated into an assembly of chambers and sensors that are connected by pumps.

17. The system of claim 16, wherein
an energy rich homogenate chamber connects to a filter chamber,
the filter chamber connects to a first biomolecule sensor,
the first biomolecule sensor connects to a photosynthesis chamber,
the photosynthesis chamber connects to a second biomolecule sensor, and
the second biomolecule sensor connects to the energy rich homogenate chamber,
wherein the photosynthesis chamber has at least one perforated, elongate chamber covered by a clear tube and having chloroplast dispersed within.

18. The system of claim 17, wherein
an input reservoir connects to the energy rich homogenate chamber,
a coulochem cell connects in between the energy rich homogenate chamber and the filter chamber,
the first biomolecule sensor connects to the energy rich homogenate chamber,
a carbon dioxide input connects to the photosynthesis chamber and adds carbon dioxide to a first end of the photosynthesis chamber, and
peristaltic pumps move material between chambers, sensors, the coulochem cell and the input reservoir.

19. A method of reducing carbon dioxide, the method comprising:
creating an energy enhanced organism strain by using a selection process applied to a first organism strain for a plurality of generations;
creating an energy rich homogenate from the energy enhanced organism strain;
pumping the energy rich homogenate from an energy rich homogenate chamber into a filter chamber to produce a filtered material;
pumping the filtered material through a first biomolecule sensor to a photosynthesis chamber;
adding a predetermined quantity of carbon dioxide to an inferior end of the photosynthesis chamber;
combining the filter material with the carbon dioxide and a chloroplast solution in the photosynthesis chamber;
reducing the quantity of carbon dioxide within the photosynthesis chamber through photosynthesis;
creating a glucose product within the photosynthesis chamber; and
pumping at least the glucose product from a superior end of the photosynthesis chamber through a second biomolecule sensor and to the energy rich homogenate chamber.

20. An interconnected photosynthesis matrix and bio-energy production system, the interconnected system comprising:
a bio-energy production system comprising
a selection process, wherein the selection process is applied to a first organism strain for a plurality of generations to create a second organism strain with enhanced energy availability, and
an extraction process, wherein the extraction process creates an energy rich homogenate from the energy enhanced organism strain; and
a photosynthesis matrix comprising
carbon dioxide, and
a chloroplast solution having homogenized plant material and an ATP solution,
wherein the ATP solution is derived from the energy rich homogenate,
wherein the photosynthesis matrix consumes the carbon dioxide and produces glucose,
wherein the glucose from the photosynthesis matrix is used as a food source for the bio-energy production system,
wherein the interconnected system is incorporated into an assembly of chambers and sensors that are connected by pumps, and
wherein one of the chambers is a photosynthesis chamber comprised of a plurality of perforated, elongate chambers each covered by a clear tube and having chloroplast dispersed within, the plurality of perforated, elongate chambers connected to a carbon dioxide input on a first end.
